(19) United States
(12) Patent Application Publication
(10) Pub. No.: US 2014/0066767 A1
(43) Pub. Date: Mar. 6, 2014

(54) SYSTEM AND METHOD FOR NOISE REDUCTION AND SIGNAL ENHANCEMENT OF COHERENT IMAGING SYSTEMS

(71) Applicants: Richard Mammone, Warren, NJ (US); Christine Podilchuk, Warren, NJ (US); Lev Barinov, North Brunswick, NJ (US); Ajit Jaoraj, Edison, NJ (US); William Hulbert, Somerset, NJ (US)

(72) Inventors: Richard Mammone, Warren, NJ (US); Christine Podilchuk, Warren, NJ (US); Lev Barinov, North Brunswick, NJ (US); Ajit Jaoraj, Edison, NJ (US); William Hulbert, Somerset, NJ (US)

(73) Assignees: Clearview Diagnostics, Inc., Warren, NJ (US); Rutgers, The State University of New Jersey, New Brunswick, NJ (US)

(21) Appl. No.: 14/016,078

(22) Filed: Aug. 31, 2013

Related U.S. Application Data
(60) Provisional application No. 61/695,722, filed on Aug. 31, 2012.
(60) Provisional application No. 61/816,581, filed on Apr. 26, 2013.

(51) Int. Cl. A61B 8/08; A61B 8/00

(52) U.S. Cl. CPC A61B 8/5269; A61B 8/0825; A61B 8/461
USPC 600/442; 600/443; 600/437

(57) ABSTRACT

A system and method of processing an ultrasound signal may include, in response to receiving a set of complex frequency samples of the ultrasound signal inclusive of content data and noise at a first noise level and being used to image an anatomical region of a body, resampling multiple subsets of complex frequency samples from the set of complex frequency samples. The resampled subsets of complex frequency samples may be resampled from a first domain into a second domain. The transformed resampled subsets of complex frequency samples may be combined in the second domain to produce a result signal with a second noise level reduced from the first noise level. An image derived from the result signal may be displayed.

### RELATED APPLICATIONS

This application claims priority to co-pending U.S. Provisional Patent Application Ser. Nos. 61/695,722 filed Aug. 31, 2012 entitled “Method for Restoring Coherent Image Degradations due to Speckle and Attenuation” and 61/816,581 filed Apr. 26, 2013 entitled “System and Method for Noise Reduction and Signal Enhancement of Coherent Imaging Systems”; the entire contents of which are hereby incorporated by reference in their entirety.

### BACKGROUND

To date, mammography remains the preferred screening tool for the timely detection of breast cancer largely because it uncovers early indicants of pathology, such as microcalcifications. Yet, the effectiveness of mammography, an ionizing and expensive modality, is profoundly diminished in women with radiographically dense breasts. The sensitivity of mammography can be as low as 48% in extremely dense breasts. For high risk women and women with dense breast tissue, ultrasound, an imaging modality that is non-ionizing, non-invasive and painless, is especially useful, but it too suffers from limitations.

Ultrasound, as with all coherent imaging systems, exhibits speckle noise, which is a granular interference that degrades the overall quality of content, such as an image, of a signal. Speckle noise reduces the contrast resolution of the image so that low-contrast lesions and small particles may be obscured within the speckle. Consequently, ultrasound images (i) inadequately detect microcalcifications, an important feature in intraductal carcinoma or ductal carcinoma in situ (DCIS) and small invasive cancer, and (ii) suffer from high rates of both false positive and false negative results. Studies show that due to the speckle artifact normally present in breast tissue, most calcifications remain occult sonographically unless present within a mass. Techniques that attempt to reduce speckle noise in ultrasound images have been a longstanding research focus and include enhanced resolution methods, compounding (averaging) methods, and post-processing (filtering) methods. While these methods achieve some reduction in speckle noise, they sacrifice the ability for an ultrasound system to visualize small details within breast tissue, which are essential for diagnosing pathology.

Recent advances in ultrasound imaging include the ability to image breast tissue at higher resolution by using higher frequency and wider bandwidth probes. These techniques can significantly enhance image resolution or quality, but they are severely constrained by signal attenuation, limiting the ultrasound's ability to sufficiently penetrate breast tissue or other organs that are deeper in the body such as liver or kidney. This attenuation diminishes the ultrasound's ability to penetrate the breast, and makes it more difficult to visualize small lesions that are deeper in the breast tissue. The ability to distinguish a complicated cyst that is less than 5 mm in diameter from a solid mass may be especially problematic, and even simple cysts can be difficult to characterize when they are deep in the breast tissue. Automatic attenuation techniques, incorporated into all conventional ultrasound hardware, achieve some benefit, but do not adequately address the issue. These techniques, known as time gain compensation (TGC) provide time-varying amplification determined by a compensation curve. Conventional ultrasound systems have attempted to build on this concept by proposing algorithms that focus on the individual compensation of each scan line. Similar to conventional TGC, these algorithms adjust for depth attenuation based only on the time of the arrival of a reflection signal, and do not take into consideration the wideband frequency response and correction due to frequency attenuation of an incident and corresponding reflection signal. These methods are limited in their ability to adequately image tissue that is deeper in the body.

Moreover, many coherent imaging systems, such as ultrasound medical imaging systems, laser imaging, sonar and synthetic aperture radar imagery (SAR), produce images that are degraded by speckle noise and attenuation effects. Speckle noise in an image can make it difficult to find small targets, such as microcalcifications, in an ultrasound breast scan since the speckle has a similar appearance to the cluster of small calcium particles that form the target of interest. Speckle noise can also make it difficult to see low contrast, small lesions, and lesion boundaries that contain characteristics that are important for diagnosis and that provide for the ability to differentiate between malignant and benign tumors.

Speckle-reduction techniques can be classified into two categories: (i) compounding methods and (ii) post-processing techniques. The compounding speckle-reduction methods include spatial and frequency compounding. These schemes rely on making separate images that have uncorrelated or partially correlated speckle patterns. These images may then be averaged to reduce the speckle, but at the expense of spatial resolution. Post-processing speckle-reduction techniques, such as median filters, Lee filters, etc., reduce speckle after the ultrasound image is formed. The engineering tradeoffs vary based on the post-processing speckle-reduction technique employed, but typically include increased contrast and reduced speckle versus edge preservation, image blurring, and image texture. Post-processing techniques, such as frequency compounding, were introduced to reduce the effect of speckle noise. Frequency compounding separates the input RF ultrasound pulse into two or more coherent frequency bands (e.g., using a series of narrowband filters) that are then noncoherently detected and summed to reduce speckle. This approach is widely used, but has the drawback that spatial resolution is limited by the smaller bandwidth used in the narrowband filters. The net effect of the resolution loss cancels the positive effect of decreased speckle in the case of detecting small lesions and microcalcifications, such as those found in breast cancer scans.

Another problem that arises in coherent imaging is image degradation due to the effects of attenuation with increasing space/time and frequency. For example, ultrasound in soft tissue applications, such as breast scans, is degraded by 1 dB/MHz/cm round trip. This attenuation decreases signal-to-noise ratio (SNR) for higher frequency components of the signal, which decreases both resolution and penetration depth.

As provided above with regard to sensing small features, such as microcalcifications, conventional imaging and processing techniques cause at least one measurement parameter to be limited such that small features may be masked by speckle and/or other noise sources.

### SUMMARY

The principles of the present invention overcome the limitation of loss in resolution while decreasing the speckle noise by using an average of reconstructed noncoherent images, each of which being formed using a random subset of frequencies that are sampled over an entire bandwidth of an image. The use of the full bandwidth maintains the resolution in the time/space domain. The random thinning of the Fourier components for each estimate provides estimates that are uncorrelated in terms of speckle, and can thus be averaged to lower the speckle seen in the compound image. Statistical resampling may be used to calculate a highly accurate sample mean and standard deviation for each time/space sample. First order statistics may be used to form a signal proportional to the original signal with reduced speckle. A filtering technique may also introduced that attenuates the speckle noise while preserving the details of the desired signal. This technique can be applied to any filter that uses estimates of signal and noise statistics in the filter design.

One embodiment of a method of processing an ultrasound signal may include, in response to receiving a set of complex frequency samples of the ultrasound signal inclusive of content data and noise at a first noise level and being used to image an anatomical region of a body, resampling multiple subsets of complex frequency samples from the set of complex frequency samples. The resampled subsets of complex frequency samples may be resampled from a first domain into a second domain. The transformed resampled subsets of complex frequency samples may be combined in the second domain to produce a result signal with a second noise level reduced from the first noise level. An image derived from the result signal may be displayed.

One embodiment of a system for processing an ultrasound signal may include a processing unit and a memory configured to store data, and in communication with the processing unit. The processing unit may be configured to, in response to receiving a set of complex frequency samples of the ultrasound signal inclusive of signal and noise at a first noise level and being used to image an anatomical region of a body, resample multiple subsets of complex frequency samples from the set of complex frequency samples. The processing unit may further be configured to transform the resampled subsets of complex frequency samples from a first domain into a second domain, combine the transformed resampled subsets of complex frequency samples in the second domain to produce a result signal with a second noise level reduced from the first noise level, and display an image derived from the result signal.

The principles of the present invention, in another embodiment, may provide for increasing the SNR of high frequency components by integrating higher Fourier components over a longer time interval while using a stepped frequency radar type of coherent imaging system. The longer integration times provide higher SNR for the higher frequency components as they are measured so an inverse filter can be used in the frequency domain without increasing the noise with increasing frequency.

Another aspect of the principles of the present invention includes calculating local attenuation differences as well as local phase properties, which can be used as an additional tool for display as an image map to aid an operator, such as an ultrasound operator, in determining characteristics of the tissue, such as fat, muscle, or cancerous areas for further evaluation. It should be understood that the principles of the present invention may be used for any signal measurement process that seeks to eliminate noise and provide high signal-to-noise ration to provide high resolution of measurement signals. Such signal measurement processes may include, but are not limited to, sonar, seismic, MRI, radar, and so on. Other non signal measurement processes, such as communications signals (e.g., satellite, mobile devices, etc.) may also use the principles of the present invention.

### BRIEF DESCRIPTION OF THE DRAWINGS

Illustrative embodiments of the present invention are described in detail below with reference to the attached drawing figures, which are incorporated by reference herein and wherein:

FIG. 1 is a block diagram of an illustrative measurement system, such as an ultrasound system, that utilizes the principles of the present invention for identifying details within an image of a medium, such as a human body;

FIG. 2 is a flow diagram of an illustrative process for correcting an image collected by a measurement system, such as that provided in FIG. 1, utilizing a resampling process in accordance with the principles of the present invention;

FIG. 3 is a flow diagram of an illustrative input signal collection process;

FIG. 4 is a flow diagram of the illustrative resampling process of FIG. 2;

FIG. 5 is a more extensive flow diagram for using resampling as provided in FIG. 5 that includes combining both a sample signal and complement of the sample signal to provide a higher resolution with better SNR than possible without using the complement of the sample signal;

FIG. 6 is a flow diagram of illustrative sub-processes that may be utilized to improve SNR of an image or communications process in accordance with the principles of the present invention;

FIG. 7 is a flow diagram of an illustrative resampling overview inclusive of sub-processes utilizing the principles of the present invention;

FIG. 8 is flow diagram of an illustrative resampling pre-processing of input signals of FIG. 7;

FIG. 9 is a flow diagram of an illustrative resampling mask generation sub-process of FIG. 7 for generating random samples of the pre-processed input signals of FIG. 8;

FIG. 10 is a flow of an illustrative magnitude signal generation sub-process of FIG. 7;

FIG. 11 is a flow diagram of an illustrative phase signal generation sub-process of FIG. 7;

FIG. 12 is an illustrative visualization sub-process that may be used to improve display of output signals for an operator of a measurement system;

FIG. 13 is a block diagram of an illustrative process for providing computer aided diagnosis for assisting an operator in determining features included within a measurement (e.g., image) captured by a measurement system;

FIG. 14 is a graph showing an illustrative A-scan measured signal inclusive of microcalcifications and noise;

FIG. 15 is a graph showing the measured signal of FIG. 14 after being processed using conventional frequency compounding techniques;

FIG. 16 is a graph showing the measured signal of FIG. 14 after being processed using the resampling process provided in FIGS. 7-11;

FIG. 17 is a zoomed-in view of a portion of the A-scan of FIG. 16;

FIGS. 18A and 18B are B-scan images of a raw ultrasound signal measuring breast tissue and attenuation-corrected ultrasound signal, respectively;

FIGS. 19A and 19B are B-scan images of a raw ultrasound signal and speckle noise corrected ultrasound signal showing how speckle noise is reduced and lesion boundaries are clearer;

FIGS. 20A and 20B are B-scan images of a raw ultrasound signal and speckle noise corrected ultrasound signal showing how speckle noise is reduced and lesion boundaries are clearer;

FIGS. 21A and 21B are B-scan images of a raw ultrasound signal and speckle noise corrected ultrasound signal showing how speckle noise is reduced and lesion boundaries are clearer;

FIGS. 22A and 22B are B-scan images of a raw ultrasound signal and speckle noise corrected ultrasound signal showing how speckle noise is reduced and lesion boundaries are clearer; and

FIG. 23 is a flow diagram of an illustrative process for resampling an ultrasound signal to reduce speckle noise.

### DETAILED DESCRIPTION

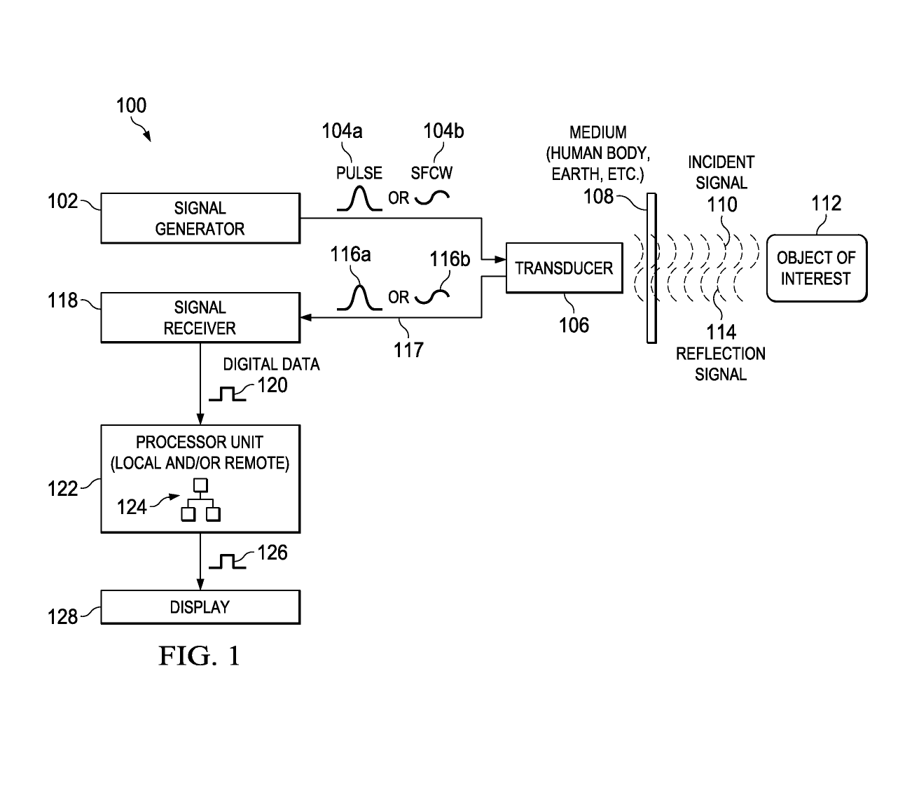

With regard to FIG. 1, a block diagram of an illustrative measurement system 100 is shown. The measurement system 100 may be an ultrasound system, seismic system, radar system, sonar system, or any other measurement system. Although principally used for measurement systems, the principles of the present invention may be utilized for communications or other systems that have noise sensitive situations that may be improved with higher signal-to-noise ratio (SNR).

The measurement system 100 may include a signal generator 102 configured to generate a pulse signal 104a or stepped frequency continuous wave (SFCW) signal 104b that is communicated to a transducer 106. Any other generated signal type that may be used for remote or non-invasive measurement or communicating information may be utilized in accordance with the principles of the present invention. For example, chirp signals, pulse signals, or other signal format may be utilized. The transducer 106, in response, may apply the generated signal 104a or 104b to a medium 108 by converting the signal 104a or 104b into an incident signal 110. The medium 108 may be a human water, earth, water (e.g., ocean), human-made structure (e.g., infrastructure, airplane fuselage, boat hull, etc.), or any other medium 108 within which the system 100 may be used to measure small features or discontinuities (or communicate and receive signals). In response to the incident signal 110 contacting an object of interest 112, a reflection signal 114 may be reflected from the object of interest 112. The object of interest 112 may be any mass, such as a tumor, hard material (e.g., microcalcification, iron ore, rock, etc.), discontinuity (e.g., crack in a structure), or otherwise from which the incident signal 110 may be reflected.

The transducer 106 (or receiver element that is independent from a transmitter element) may receive the reflected signal 114. Received signal 116a or 116b, depending on the type of incident signal 110, may be communicated via a transmission line 117 to a signal receiver 118. The received signal 116a or 116b may be analog signals or digital signals if the transducer 106 is configured to convert the reflection signal 114 into a digital signal prior to communicating to the signal receiver 118 (or other electronics). The transmission line may be copper, optical, or wireless, as understood in the art. In response, the signal receiver 118 may be configured to perform a processing operation to convert the received signal 116a or 116b into a digital data signal 120 using an analog-to-digital conversion process, as understood in the art. The digital data signal 120 may be formatted in any communications protocol, as understood in the art. The signal receiver 118 may also be configured to perform conventional signal processing in performing the digitization process.

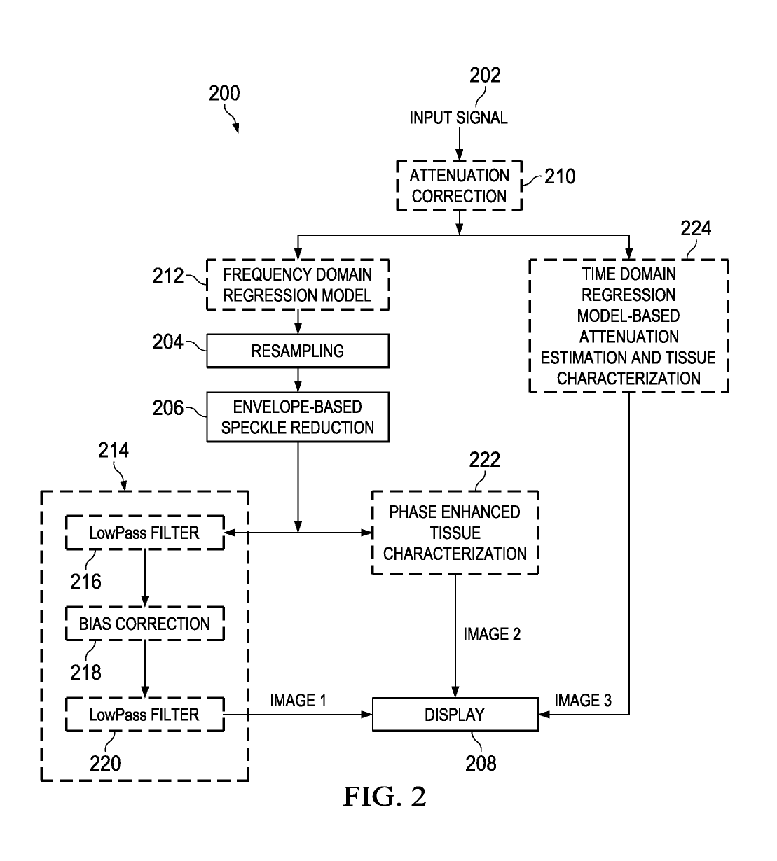

A processor or processing unit 122, which may be formed of one or more computer processors, that is local and/or remote from the signal receiver 118 may be configured to execute software 124. The computer processor(s) may be general computer processor(s), digital signal processor(s), image processor(s), and/or other processor(s) or processing device(s) capable of executing the software 124. The software 124 may be configured to perform a resampling process, among other functionality, as further provided herein (see, for example, FIGS. 2-5). The resampling process may be a random resampling process. A memory (not shown) may be utilized to store data and/or programming instructions for use by the processor unit 122 in processing the digital data signal 120. In the case of the digital data signal 120 representing image data, such as ultrasound data, the processor unit 122 may generate an output signal 126 in the form of an image using a predetermined format and protocol, as understood in the art. If the digital data signal 120 represents another type of data, such as audio, then the output signal 126 may be in the format and protocol of that other type of data. Although not shown, amplifier or other signal conditioning electronics may be utilized to condition the output signal 126 prior to being displayed on an electronic display 128 or output by another electronic device (e.g., speaker), as understood in the art. In the event that the output signal 126 is being communicated to another processor, such as a processor used by a targeting system to automatically guide a larger system (e.g., unmanned vehicle) in which the system 100 is incorporated, then the output signal 126 may be formatted for such a communication. As will further be described herein, the output signal 126 may result in a signal with improved signal-to-noise levels as a result of lowering speckle noise to accommodate for identifying an object of interest that would otherwise be difficult to identify with higher signal-to-noise levels.

In one embodiment, the signal generator 102, transducer 106, and signal receiver 118 are packaged in a single unit, such as a handheld unit. Alternatively, the transducer 106 may be independent from the signal generator 102 and signal receiver 118. The processor unit 124, in being local, may be packaged in the same housing (not shown) as the signal receiver, signal generator, and/or transducer. The processor unit 122 may be considered remote if the signal receiver 118 is in a separate housing from the processor unit 122. In one embodiment, the signal receiver may be separated via a communications network, such as a mobile and/or wide area network (WAN), such as the Internet, and the digital data signal 120 may be communicated over the communications network utilizing one or more communications protocols, as understood in the art. As an example, the signal generator, transducer, and signal receiver may be packaged in a housing and connected to a mobile device, such as a mobile smart phone, tablet computer, portable computer, or other mobile electronic device, and the processor unit 122 may be operated on a server or other computing system operating on the communications network (e.g., cloud computing). The electronic display 128 may be any electronic display, such as a tablet computer electronic display, that is capable of displaying output data 126. Of course, any other electronic device other than an electronic display may be utilized depending on the format of the output data 126, environment in which the system 100 is being utilized, and so on.

With regard to FIG. 2, a flow diagram of an illustrative process 200 for correcting an image collected by a measurement system, such as that provided in FIG. 1, utilizing a resampling process in accordance with the principles of the present invention is shown. The process 200 may provide for noise reduction and signal enhancement of a coherent image or other data format (e.g., radar, sonar, etc.). The process 200 may include an input signal 202 that may be in the form of a reflection signal or incident signal depending on the type of system and data being collected. The input signal 202 may be a digital signal that has been formatted for the process 200. As shown, there are a number of functions or modules that have dashed lines, which means that the functions are optional, and a number of functions or modules having solid lines, which means that the functions are to be utilized. Although the functions are shown in a particular order, it should be understood that the order of the functions may be altered, use of the functions may be optional, and that the functions may also be repeated. The functions or modules provided in FIG. 2 and other figures may be configured as hardware and/or software, as understood in the art.

Resampling & Envelope-Based Speckle Reduction

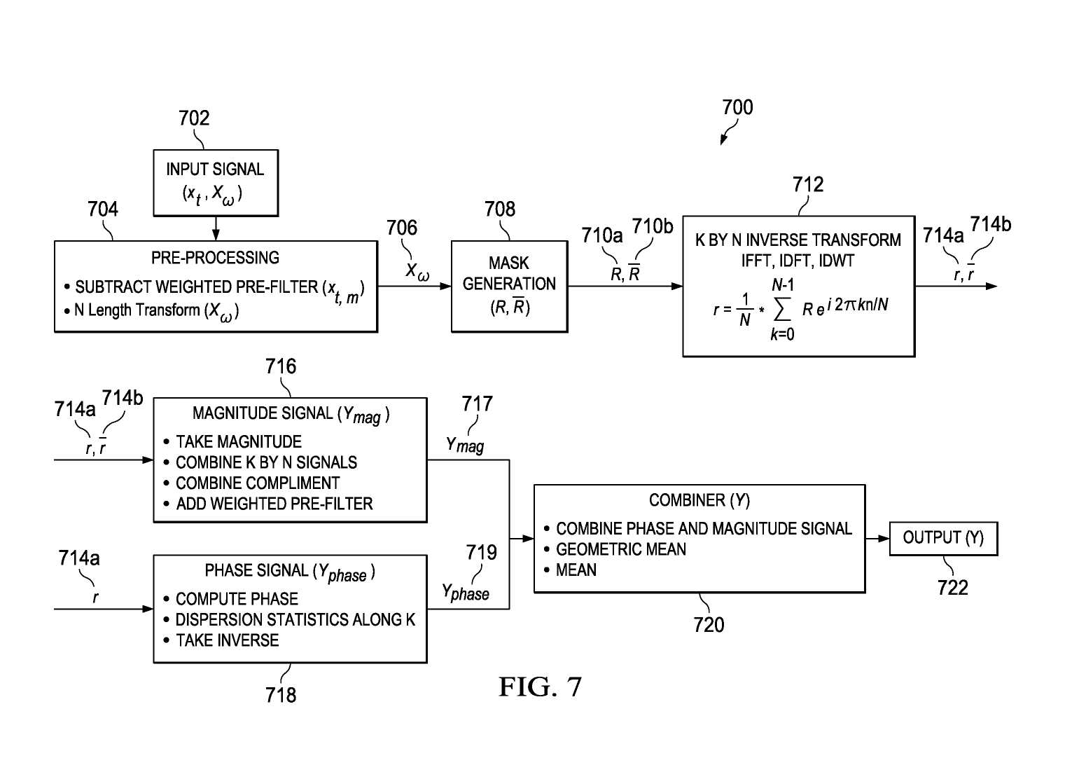

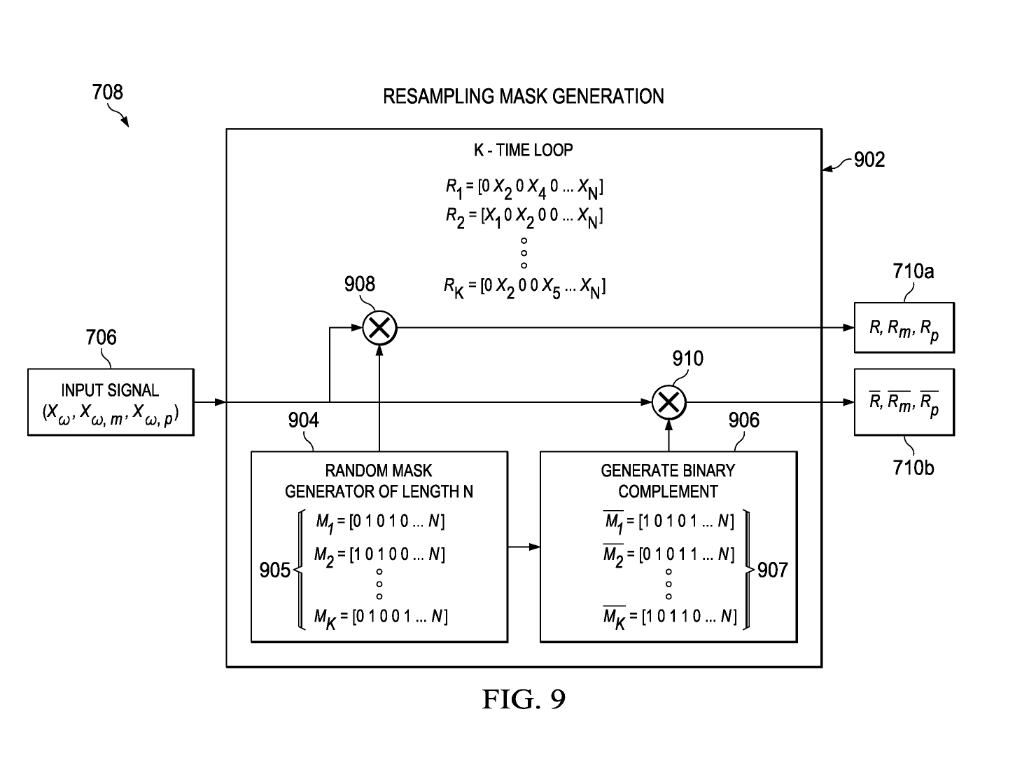

Resampling module 204 and envelope-based speckle reduction module 206 provide for sampling a set of complex frequency samples into multiple subsets of complex frequency samples, as further described herein with respect to FIGS. 7-11. Ultrasound and other sensing techniques often suffer from noise and other sensing limitations, as understood in the art. One type of noise is known as speckle noise. Conventional sensing systems, such as ultrasound systems, typically perform frequency compounding utilizing narrowband filters (e.g., a series of non-overlapping narrowband filters), envelope detection, and log compression functionality. While frequency compounding has been held up as a signal improvement for certain types of noise, such processing actually adds little, if any, improvement in resolution with regard to speckle noise.

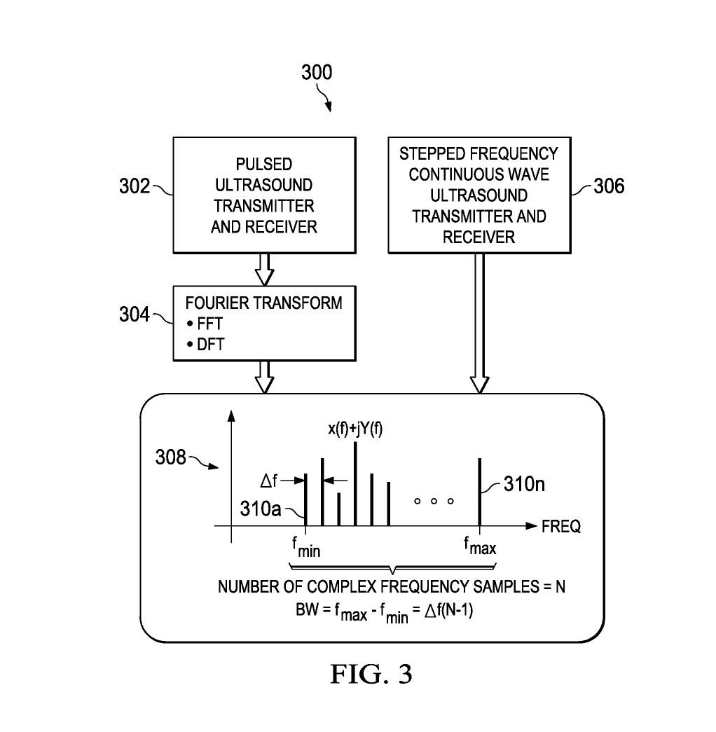

In accordance with the principles of the present invention, a statistical sampling method may be used to process a radio frequency (RF) return signal (or other reflection signal) from a pulse echo signal or a continuous wave (CW) signal. A pulse return can be transformed into the frequency domain by using a fast Fourier transform (FFT) or similar algorithm, as understood in the art. The CW return signal can be represented in the frequency domain by a vector containing the magnitude and phase or in-phase and quadrature components of the return signal at each frequency. The resulting frequency domain signal includes N complex numbers at a set of frequencies fᵢ, i=0, 1, 2, . . . N−1. A block diagram of the front-end of the statistical sampling method for coherent signal enhancement is shown in FIG. 3. The frequency components may be randomly sampled so, for example, 50% are removed at random by using a uniform random number generator that generates a number between 0 and 1 and setting to zero any ith component X(fᵢ) if the ith random number is chosen as below 0.5. The remaining 50% of X(fᵢ)'s are inverse Fourier transformed to obtain a time envelope. This is repeated M times, and all M time envelopes are averaged to reduce speckle variations while leaving the signal unchanged.

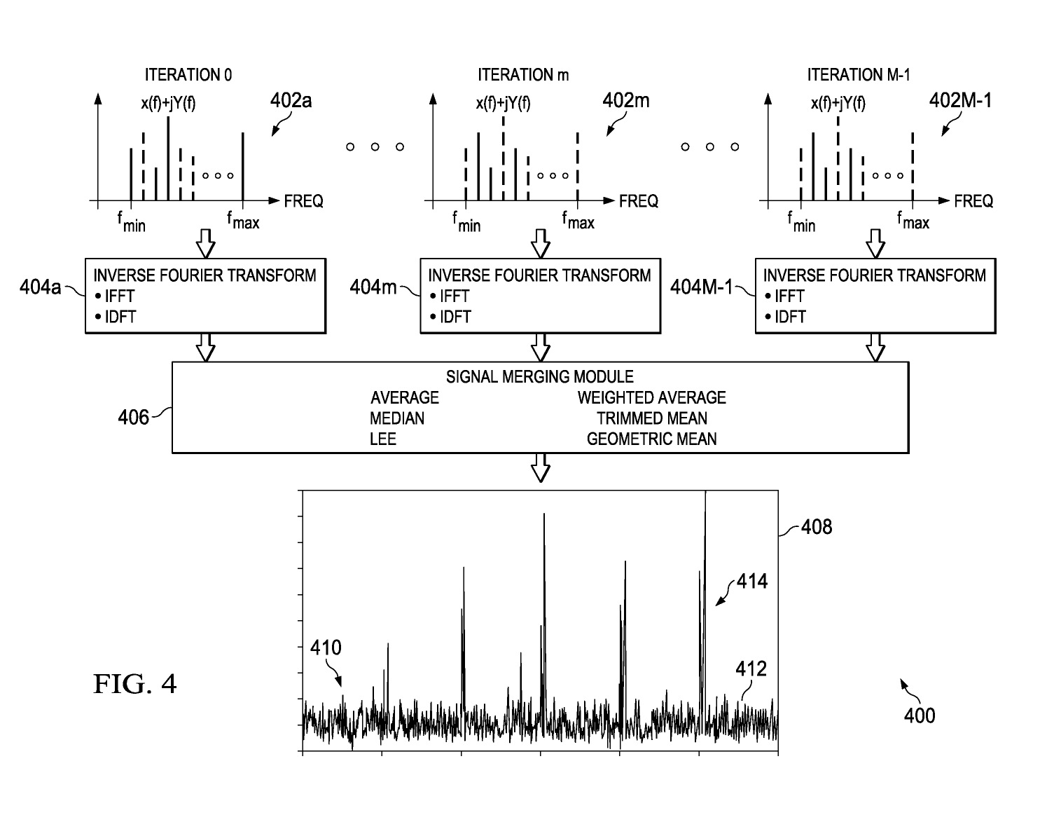

The block diagram illustrating the steps of the statistical sampling method for coherent signal enhancement is shown in FIG. 4. The complex frequency coefficients X(fᵢ)+jY(fᵢ) may be randomly subsampled and the coefficients, which are zeroed out, are shown in light gray, while the complex coefficients that are kept are illustrated in black. This random subsampling of the coefficients is performed M times and the inverse Fourier Transform is computed for the M subsampled sequences. Although a uniform random number generator may be utilized for selecting which samples and coefficients to zero out, it should be understood that alternative functions and/or techniques may be utilized in the resampling process.

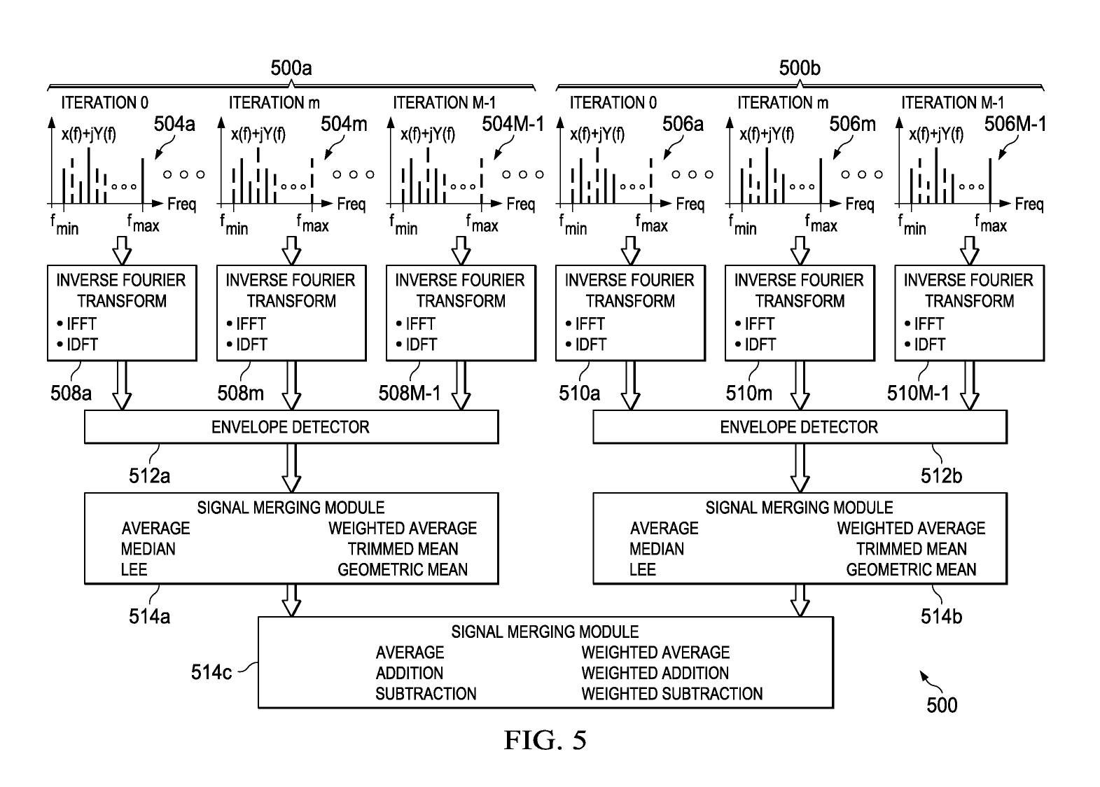

A merging or averaging module may be used to merge the M time/spatial domain signals and the output is the enhanced time-domain signal with reduced speckle and improved SNR. An alternative statistical sampling technique for signal enhancement and speckle noise reduction is illustrated in FIG. 5. This method is similar to that described above and shown in FIGS. 3 and 4 with the exception that the complement to the subsampled Fourier samples is also computed for each of the M sequences and all M sequences are merged in some way. The complement to any subsampled spectrum includes zeroing out all the frequency components that are kept in the original subsampled sequence and keeping all the frequency components that were originally zeroed out.

The union of the subsampled sequence and the complement to the subsampled sequence is the entire spectral sequence while the intersection of the two sequences is the null set. As an example, if 60% of the samples are kept in the original subsampled spectrum, then 40% of the samples are kept in the complement. The subsampled Fourier samples and the complement to the subsampled Fourier samples may also have some overlap in Fourier samples that each one keeps or discards. In other words, the union of the subsampled sequence and the complement to the subsampled sequence can be less than the entire spectral sequence while the intersection of the two sequences may not be the null set. For this case, the total percentage of the original subsampled spectrum and complement do not have to add up to 100% so that if 60% of the samples are kept in the original subsampled spectrum, then any number between 0 and 100% can be kept in the complement. It should also be understood that while subsampling across the entire frequency spectrum of the original samples provides for full resolution and noise reduction that subsampling over less than the entire bandwidth (e.g., not sampling over certain known content data frequencies) may provide substantially full resolution and noise reduction (e.g., >99% resolution and <0.1 dB difference in noise floor). The final step of the process of FIG. 5 may be to merge the resulting envelope from subsampling with its complement after merging each over M iterations. The final merging step may be an average, sum, subtraction, weighted average, or other merging function between two time series. In one embodiment, subtraction of the complement from the subsampled original may be performed.

Attenuation Correction

An attenuation correction module 210 may be performed on the raw input signal to correct for attenuation of the measured ultrasound signal due to depth of tissue into which the ultrasound signal is injected. Over the last several years, ultrasound quality has improved due to increased resolution obtained from probes that are operating at higher frequencies. However, high frequency signals result in greater signal attenuation as the ultrasound signal travels through the breast tissue. This attenuation diminishes the ability for an ultrasound system to penetrate breast tissue, and makes it more difficult to visualize small lesions that are deeper in the breast tissue. The ability to distinguish a complicated cyst that is less than 5 mm from a solid mass may be especially problematic, and even simple cysts can be difficult to characterize when they are deep in the breast tissue. Although current US devices are equipped with circuitry to provide time-gain compensation (TGC), which is a time-varying amplification, this does not compensate for attenuation due to operating frequency, which is especially important in high frequency, wide bandwidth transducers used for many imaging applications today. The principles of the present invention provide for a more accurate attenuation correction due to distance into the tissue and frequency, which allows for (i) better visualization of lesions or other features that are deeper in breast tissue and (ii) the ability for more accurate diagnosis.

All conventional ultrasound systems are equipped with circuitry that performs time-gain compensation. In practice, most systems have additional slide potentiometers, which allow the gain to be determined interactively by the operator. One such slide potentiometer is a frequency dependent slid potentiometer. By providing a slide potentiometer, the user may be permitted to manually adapt the system to special circumstances to provide more or less gain so that subtle features can be seen in the images.

Attenuation of an ultrasound signal exponentially increases as a function of depth i and frequency f. The ultrasound signal attenuation is given by:

Xₐₜₜₑₙ(i,f)=e^{<αfi} sin(i,f) (1)

where α is the attenuation coefficient, which is approximately 0.5 dB/cm/MHz for breast tissue or 1 dB/cm/MHz round trip, i is distance traveled, and f is frequency. The attenuation can be corrected approximately by selecting I_{avg}=(iₘₐₓ−iₘᵢₙ)/2 and f_{avg}=(fₘₐₓ−fₘᵢₙ)/2 and multiplying the frequency domain attenuated signal by:

X_{fcorr}(f)=Xₐₜₜₑₙ(f)*e^{αfI}^{_{avg}} (2)

to correct for attenuation in the frequency domain, where X_{fcorr} is the frequency corrected signal.

Similarly the time/space domain signal can be multiplied by:

X_{icorr}(i)=Xₐₜₜₑₙ(i)*e^{αiW}^{_{avg}} (3)

to correct for attenuation as a function of distance, where X_{icorr} is the distance corrected signal. Both operations can be performed sequentially to get the final result with attenuation correction for frequency and distance, that is,

X̂*(i)=X_{icorr}(i)IFFT{[X_{fcorr}(ω)]}. (4)

The total attenuation correction incorporates both time and frequency corrections using the mid-point for distance and frequency. For example, a breast cancer ultrasound that operates over a bandwidth from 5 MHz to 15 MHz has a mid-frequency of 10 MHz and a depth from 0 to 10 cm has a mid-range value of 5 cm.

However, the attenuation is a function of distance and frequency that is not separable so the approach shown in Equation (5) below becomes more inaccurate as frequencies get further away from ω_{avg} and distances get further away from I_{avg}. In order to provide useful attenuation over a broad band of frequencies and distances, an exact solution to the exponential attenuation in frequency and distance is proposed as well as efficient and stable matrix inversion methods.

An attenuation matrix A is derived and an inverse of this matrix provides the attenuation correction matrix to be applied to the measured ultrasound signal. The Discrete Fourier Transform (DFT) is defined as:

$$X ( {f}_{n} ) = {\sum }_{m = 0}^{M - 1} x ( {t}_{m} ) {e}^{- i ( \frac{2 π}{N} ) {f}_{n} {t}_{m}} for 0 \leq n \leq N - 1 \qquad ( 5 )$$

and the Inverse DFT (IDFT) is given by:

$$x ( {t}_{m} ) = \frac{1}{N} {\sum }_{n = 0}^{N - 1} x ( {f}_{n} ) {e}^{i ( \frac{2 π}{N} ) {f}_{n} {t}_{m}} for 0 \leq m \leq M - 1 \qquad ( 6 )$$

The attenuated received signal can be expressed as a modified IDFT, where each coefficient is scaled by the exponential attenuation factor that gets damped in frequency and distance, that is,

$${x}_{atten} ( {t}_{m} ) = \frac{1}{N} {\sum }_{n = 0}^{N - 1} x ( {f}_{n} ) {e}^{i ( \frac{2 π}{N} ) {f}_{n} {t}_{m}} {e}^{- α {f}_{n} {t}_{m}} for 0 \leq m \leq M - 1 \qquad ( 7 )$$

Converting this expression to matrix notation yields:

$$\begin{matrix}[ \begin{matrix}{x}_{atten} ( {t}_{0} ) \\ {x}_{atten} ( {t}_{1} ) \\ ⋮ \\ {x}_{atten} ( {t}_{N - 1} )\end{matrix} ] = \frac{1}{N} [ \begin{matrix}{e}^{i ( \frac{2 π}{N} ) {f}_{0} {t}_{0}} {e}^{- α {f}_{0} {t}_{0}} & {e}^{i ( \frac{2 π}{N} ) {f}_{1} {t}_{0}} {e}^{- α {f}_{1} {t}_{0}} & … & {e}^{i ( \frac{2 π}{N} ) {f}_{N - 1} {t}_{0}} {e}^{- α {f}_{N - 1} {t}_{0}} \\ {e}^{i ( \frac{2 π}{N} ) {f}_{0} {t}_{1}} {e}^{- α {f}_{0} {t}_{1}} & {e}^{i ( \frac{2 π}{N} ) {f}_{1} {t}_{1}} {e}^{- α {f}_{1} {t}_{1}} & … & {e}^{i ( \frac{2 π}{N} ) {f}_{N - 1} {t}_{1}} {e}^{- α {f}_{N - 1} {t}_{1}} \\ ⋮ & ⋮ & ⋮ & ⋮ \\ {e}^{i ( \frac{2 π}{N} ) {f}_{0} {t}_{N - 1}} {e}^{- α {f}_{0} {t}_{N - 1}} & {e}^{i ( \frac{2 π}{N} ) {f}_{1} {t}_{N - 1}} {e}^{- α {f}_{1} {t}_{N - 1}} & … & {e}^{i ( \frac{2 π}{N} ) {f}_{N - 1} {t}_{N - 1}} {e}^{- α {f}_{N - 1} {t}_{N - 1}}\end{matrix} ] [ \begin{matrix}x ( {f}_{0} ) \\ x ( {f}_{1} ) \\ ⋮ \\ x ( {f}_{N - 1} )\end{matrix} ] \\ or & ( 8 ) \\ {X}_{atten} ( t ) = A X ( f ) & ( 9 )\end{matrix}$$

where A is the attenuation matrix that represents the attenuation-scaled IDFT Taking the DFT of both sides yields:

Xₐₜₜₑₙ(f)=(DFT(A))X(f). (10)

Xₐₜₜₑₙ(f)=BX(f) (11)

Combining the DFT matrix and the A matrix into matrix B and taking the inverse yields:

X*(f)=⁻¹Xₐₜₜₑₙ(f) (12)

where X*(f) is the attenuation-corrected signal in the frequency domain. Any conventional matrix inversion technique could be used, such as Gaussian elimination, eigen decomposition, Cholesky decomposition or for a rank deficient or ill-conditioned matrix, the Moore-Penrose pseudoinverse. In practice, several properties of the signal may be exploited in order to derive an efficient and stable matrix inversion. In particular, by defining B as DFT(A) and solving for B-1 allows for reduction of the dimensionality of the matrix B by deleting any rows that correspond to very low values in the frequency components without sacrificing accuracy. The number of rows to be deleted can be determined by using conventional techniques, such as picking a threshold, where any frequency component that falls below the threshold is zeroed out. The following properties may be used to find an efficient and stable matrix inversion:
(i) The rows and columns in B may be removed that correspond to very high frequency components are close to zero.
(ii) Any attenuation coefficients that are close to zero using standard thresholding techniques or by determining the condition number of the matrix may be lower bounded in order to provide a more stable inverse and to reduce the amplification of noise when the signal strength is very weak.
(iii) The attenuation matrix inversion may be computed once and used to correct for attenuation effects on measured signals. This matrix needs to be updated only if the operational frequency range or distance range changes.
(iv) Attenuation correction, where the time and frequency domain signals are multiplied by exponential with the mean range and mean frequency of the imaging system.
(v) The frequency domain correction may be performed first followed by the time domain correction.
(vi) The time correction may be performed first followed by the frequency domain correction.
(vii) The frequency components may be integrated longer for higher frequencies in a Stepped Frequency Continuous Wave (SFCW) radar system to compensate the increase in noise levels for the attenuation correction factors amplification of high frequency components.

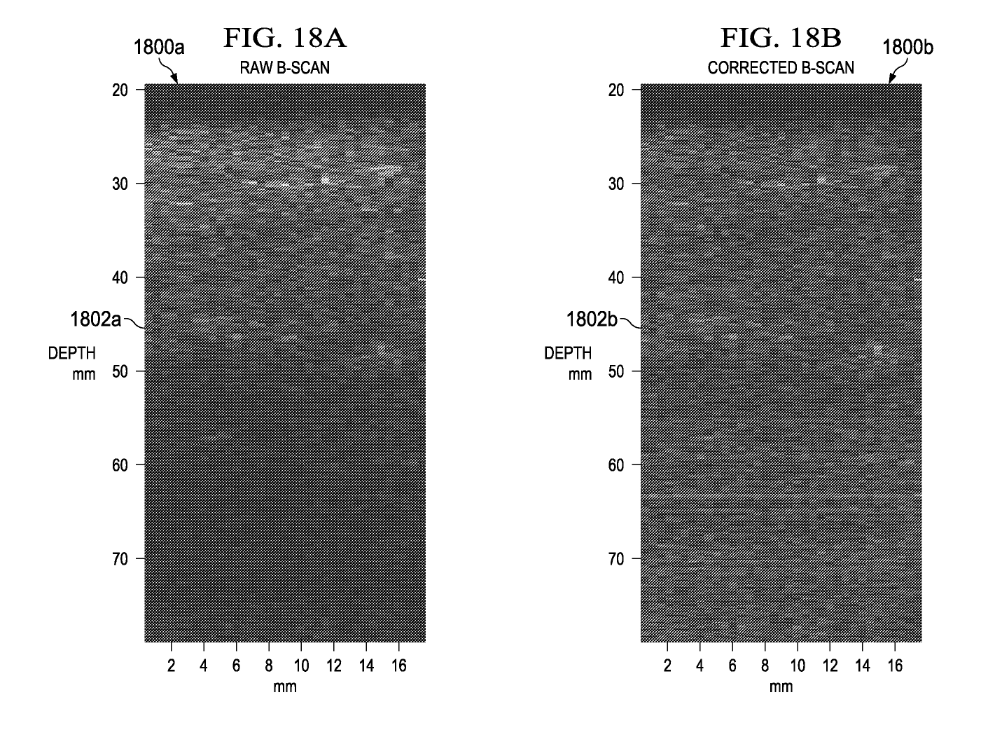

With regard to FIGS. 18A and 18B, B-scan images 1800a and 1800b of a raw ultrasound signal measuring breast tissue and attenuation-corrected ultrasound signal, respectively, are shown. It should be understood that the medium may be tissue other than breast tissue and show similar attenuation characteristics, but possibly at different depths, as provided in these B-scan images 1800a and 1800b, due to different tissue density and/or other factors, as understood in the art. Still yet, if the images are taken of other medium, such as water or earth, then, again, the attenuation characteristics may be the same or similar, but occur at different depths. As shown in FIG. 18A, below a depth of 50 mm in region 1802a, the image is dark, which indicates an attenuation of incident and/or reflection ultrasound signals. However, as a result of applying the attenuation correction function 210, the region 1802b of FIG. 18B is shown to include useful data that can be measured to identify objects of interest, such as microcalcification, at depths that are below those that could otherwise be detected without the use of the attenuation correction function 210.

Frequency Domain Regression Model

A frequency domain regression model module 212 may provide for regression analysis, which is a statistical technique for estimating the relationships among variables. Regression analysis includes many techniques for modeling and analyzing several variables when the focus is on the relationship between a dependent variable and one or more independent variables. Regression analysis, where a linear or nonlinear model is fit to observed data, can be applied to prediction, and these models can be used to separate observed noisy data into predictable or desirable components and unpredictable or noisy components. For coherent imaging techniques that suffer from speckle noise, regression analysis can be used to determine a model of the predictable or desired part of the observed signal and separate the desired part of the observed signal from the unpredictable components that are due to speckle and other noise components corrupting the measurements. Fully developed speckle is known to have a Rayleigh distribution. Complex numbers whose real and imaginary parts have independent and identically distributed random variables (i.i.d). Gaussian distributions with zero mean and equal variance result in a Rayleigh distribution on the envelope or magnitude of the signal that is:

R=\sqrt{X²+Y²} (13)

X˜N(0,σ⁻²) (14)

Y˜N(0,σ²) (15)

A Gaussian distribution remains Gaussian after applying a unitary transformation on the Gaussian distribution so that Gaussian noise in the time or spatial domain is Gaussian in the Fourier or frequency domain. Regression analysis may be applied to the measured signal from an ultrasound probe or other coherent imaging modality to obtain a linear or nonlinear model of the observed signal in order to apply prediction to the observed signal components and remove the unpredictable portion of the signal containing speckle or other noise sources from the predictable portion containing the desired signal for viewing and diagnosis.

A regression model is expressed as:

Y=f(X,A) (16)

or

E(Y/X)=f(X,A) (17)

where the unknown parameter to be solved is A that could be a scalar term or vector, independent variables are given by X, and dependent variable is Y. If A is a vector of length p, a generalized linear regression model can be expressed as:

yᵢ=a₀+a₁xᵢ₁+a₂xᵢ₂+ . . . aₚxᵢₚεᵢ (18)

where xij is the ith observation of the jth independent variable.

A simple linear regression is a first order linear predictor expressed as:

y=a₀+a₁x+ε (19)

y=ŷ+ε (20)

where x is the independent variable used to predict the dependent variable y, ŷ is the predicted (desirable) signal and E is the unpredictable (undesirable) noise term. A multiple linear regression is a regression model that predicts the dependent variable y from p independent variables x₁, x₂, . . . , xₚ≡X and

y=a₀+A^{T}X+ε (21)

Linear prediction of an autoregressive process is expressed stated as:

ŷn/n−1=−[a₁yₙ₋₁+a₂yₙ₋₂+a₃yₙ₋₃+ . . . +aₚyₙ₋ₚ] (22)

yₙ=ŷₙ+εₙ (23)

where yₙ is predicted from the last p samples [yₙ₋₁, yₙ₋₂, yₙ₋ₚ]. The prediction coefficients can be calculated by solving the normal equations expressed as:

$$[ \begin{matrix}{R}_{yy} ( 0 ) & {R}_{yy} ( 1 ) & {R}_{yy} ( 2 ) & … & {R}_{yy} ( p ) \\ {R}_{yy} ( 1 ) & {R}_{yy} ( 0 ) & {R}_{yy} ( 1 ) & … & {R}_{yy} ( p - 1 ) \\ {R}_{yy} ( 2 ) & {R}_{yy} ( 1 ) & {R}_{yy} ( 0 ) & … & {R}_{yy} ( p - 2 ) \\ … & … & … & … & … \\ {R}_{yy} ( p ) & {R}_{yy} ( p - 1 ) & {R}_{yy} ( p - 2 ) & … & {R}_{yy} ( 0 )\end{matrix} ] [ \begin{matrix}1 \\ {a}_{1} \\ {a}_{2} \\ … \\ {a}_{p}\end{matrix} ] = [ \begin{matrix}{σ}_{ε}^{2} \\ 0 \\ 0 \\ … \\ 0\end{matrix} ] \qquad ( 24 )$$

Standard techniques for solving for the prediction coefficients aᵢ can be used such as the autocorrelation, covariance or Burg's Method. The ensemble of autocorrelations in Equation (24) is replaced with sample correlations from N measurements of yₙ.

Once the prediction coefficients are estimated from the N samples, the following steps may be performed:

ŷₙ=−[a₁yₙ₋₁+a₂yₙ₋₂+a₃yₙ₋₃+ . . . +aₚyₙ₋ₚ] for 0≦n≦N−1 (25)

This method may be applied recursively to obtain better separation of the signal and noise and a better signal-to-noise ratio on the predicted signal that is:

$$\begin{matrix}{\hat{y}}_{n}^{1} = - [ {a}_{1} {y}_{n - 1} + {a}_{2} {y}_{n - 2} + {a}_{3} {y}_{n - 3} + … + {a}_{p} {y}_{n - p} ] for 0 \leq n \leq N - 1 & ( 26 ) \\ {\hat{y}}_{n}^{i} = - [ {a}_{1} {\hat{y}}_{n - 1}^{i - 1} + {a}_{2} {\hat{y}}_{n - 2}^{i - 1} + {a}_{3} {\hat{y}}_{n - 3}^{i - 1} + … + {a}_{p} {\hat{y}}_{n - p}^{i - 1} ] for 0 \leq n \leq N - 1 , 2 \leq i \leq M & ( 27 )\end{matrix}$$

The linear prediction may be recursively applied M times to the predicted estimates from the previous iteration. This method may be applied to the signal in the Fourier or time/space domain as well as to real and complex numbers, where the prediction filter is applied separately to the real and imaginary part of the signal. Regression analysis can be applied to all the observed data to find one linear model or if the data is more complex, the data can be subdivided into smaller regions where a linear model can better represent and predict the signal. Recursive partitioning of the data using prediction trees may be applied to complex data in order to provide a more complex nonlinear model using linear models over partitions of data that can be modeled with a linear model. Prediction models could be applied to the real and imaginary Fourier components of the signal in order to minimize the effect of speckle and other noise by separating the predictable signal through the linear model from the unpredictable part, which is the observed signal minus the predicted signal. Additional features may include:
(i) The regression model being applied to the received Fourier domain ultrasound signal.
(ii) The regression model being applied to a subset of the samples.
(iii) The regression model being applied separately on the real and imaginary components of the signal.
(iv) The regression model is a pth order model, which may be applied to predict one or more samples.
(v) The regression model may be applied more than once in order to minimize speckle noise and increase signal-to-noise ratio and contrast-to-noise ratio (CNR).

Lowpass Filter and Bias Correction

A lowpass filter and bias correction module 214 may include a pre-lowpass filter module 216, bias correction module 218, and post-lowpass filter module 220 to perform bias correction. Resampling introduces a bias due to the averaging of the frequency subsampled envelope of the signal in the time/space domain. The bias can be corrected in several ways. This bias correction may include a pre- and post-lowpass filter. The lowpass filter may be any one of many filters that are well known in the art that attenuate the high frequency components of the signal and retain the low frequency components. One low pass filter embodiment may be a Weiner Filter.

y=a₀+A^{T}X+ε (28)

The Weiner filter or any other filter that incorporates the mean and variance of the signal in order to describe or design the filter can further incorporate the method of resampling described above in order to obtain a better estimate of the mean and variance in the filter design.

The bias correction may be combined with a preprocessing lowpass filter, a post-processing lowpass filter, both, or neither. The bias correction on the resampled and speckle reduced output y* can be obtained by finding the maximum value of the unprocessed input y and determining the bias error e_{b} based on a difference between the original max value of y and the resampled and processed value of y* that is:

max{y}=y(t_{MAX}) (29)

e_{b}=y(t_{MAX})−y*(t_{MAX}) (30)

y_{bc}*=y*(t_{MAX})+e_{b} (31)

where y_{bc}* is the bias corrected resampled signal. An alternate bias correction method may utilize Gram Schmidt orthonormalization in order to add the bias correction or error term.

Phase Enhanced Tissue Characterization

A phase enhanced tissue characterization 222 may provide for creating an additional image to display the local tissue characteristics by using the resampling technique described above to determine local phase variance of the samples across the frequency subsampled ensembles. Instead of calculating the magnitude, which is defined as:

R=\sqrt{y_{R}²+y₁²} (32)

The phase may be calculated by:

$$θ = {tan}^{- 1} ( \frac{{y}_{I}}{{y}_{R}} ) \qquad ( 33 )$$

Techniques known in the art have all focused on using the magnitude of the signal and the fact that fully developed speckle has a constant mean over standard deviation ratio, μ/σ, of 1.91 in order to differentiate speckle noise from signal plus speckle noise. Properties of the phase may be used in order to differentiate tissue features of interest from speckle noise. Fully developed speckle with a Rayleigh distribution may contain phase with a uniform distribution between −π and π. Areas that are predominantly speckle noise may be expected to have a wider phase distribution or larger variance and standard deviation of the phase component, while areas that contain signal due to lesions or small particles generally have a narrower phase distribution or smaller variance and standard deviation. The variance of the phase across the ensemble resampled signal can be determined at each location or time sample, that is:

σ²(t)=Σᵢ₌₁^{M}(θᵢ(t)−μ_{θ}_{ᵢ})² t=1,2, . . . , N (34)

where M is the number of resampled sequences and N is the number of samples in each sequence. The variance is large for areas that are due to speckle noise and small for areas that are due to signal and speckle noise. The variance may be used to identify areas due to speckle and areas due to actual signal. One way of displaying the phase characteristics of the measured signal is to display the following:

$$\begin{matrix}{y}_{displayed} ( t ) = ( \frac{1}{σ ( t )} ) & t = 1 , 2 , … , N\end{matrix} \qquad ( 35 )$$

where large values of the standard deviation corresponding to areas of speckle noise may be displayed as small values (or black regions in a gray scale image) and large values of the standard deviation may be displayed as bright areas corresponding to the desired signal. An alternate way of using the phase to display the different local tissue characteristics is by displaying the following:

$${y}_{displayed} ( t ) = ( \frac{1}{{F}_{M} \{ \frac{{θ}_{O} ( t ) + 2 π}{{θ}_{I} ( t ) + 2 π} - 1 \}} ) \qquad ( 36 )$$

where F_{M} is a median filter, θ_{O} is the resampled phase and θ_{I} is the original phase. Any statistical properties of the resampled phase and original phase can be used to differentiate the features of interest which may be lesions or small microcalcifications from speckle noise.

Time-Domain Regression Model for Tissue Characterization

A time domain regression model-based attenuation estimation and tissue characterization module 224 may be used as a diagnostic tool to characterize tissue types, such as normal tissue and cancerous tissue. An output from the module 224 may be a third image and displayed on the display 208. Ultrasound attenuation parameters are closely related to the type and the pathological state of the tissue. Therefore, the estimated attenuation parameters can be used as a feature in quantitative tissue characterization. Ultrasound attenuation is also an important factor affecting spatial resolution of ultrasound images since the higher ultrasonic frequencies are attenuated more than the lower ones. Ultrasound attenuation estimation can be used to identify the pathology of the tissue and could be used as a diagnostic tool to identify areas that are highly indicative of cancerous tissue. A color map or other visual map can be overlayed on the ultrasound image to identify the different attenuation factors indicative of the pathological state of the tissue. The regression model described above may be used in the time domain in order to identify attenuation due to different tissue types. The regression model is computed over smaller blocks of data where the block size could be fixed in advance or the block size may be adapted by using a conventional segmentation technique to identify uniform areas in the ultrasound image. A pth order regression model may be computed as:

ŷn/n−1=−[a₁yₙ₋₁+a₂yₙ₋₂+a₃yₙ₋₃+ . . . aₚyₙ₋ₚ] (37)

yₙ=ŷhd n+εₙ (38)

where yₙ is predicted from the last p samples [yₙ₋₁, yₙ₋₂, . . . , yₙ₋ₚ]. The prediction coefficients can be calculated by solving the normal equations expressed as:

$$[ \begin{matrix}{R}_{yy} ( 0 ) & {R}_{yy} ( 1 ) & {R}_{yy} ( 2 ) & … & {R}_{yy} ( p ) \\ {R}_{yy} ( 1 ) & {R}_{yy} ( 0 ) & {R}_{yy} ( 1 ) & … & {R}_{yy} ( p - 1 ) \\ {R}_{yy} ( 2 ) & {R}_{yy} ( 1 ) & {R}_{yy} ( 0 ) & … & {R}_{yy} ( p - 2 ) \\ … & … & … & … & … \\ {R}_{yy} ( p ) & {R}_{yy} ( p - 1 ) & {R}_{yy} ( p - 2 ) & … & {R}_{yy} ( 0 )\end{matrix} ] [ \begin{matrix}1 \\ {a}_{1} \\ {a}_{2} \\ … \\ {a}_{p}\end{matrix} ] = [ \begin{matrix}{σ}_{ε}^{2} \\ 0 \\ 0 \\ … \\ 0\end{matrix} ] \qquad ( 39 )$$

Standard techniques for solving for the prediction coefficients aᵢ in the normal equation may be used, such as the autocorrelation, covariance or Burg's Method. The ensemble autocorrelations in Equation (39) are replaced with sample correlations from N measurements of yₙ. Here, the autoregressive model or all-pole model can be expressed as:

$$\begin{matrix}y ( n ) = {\sum }_{k = 1}^{p} {a}_{k} y ( n - k ) \\ or & ( 40 ) \\ Y ( z ) = \frac{1}{{\sum }_{0}^{p} {a}_{k} {z}^{- k}} = {\prod }_{0}^{p - 1} {( z - {b}_{k} )}^{- 1} & ( 41 )\end{matrix}$$

The poles and phase calculated over each region or block of samples are used to determine the attenuation or change in attenuation that characterizes the tissue type. Several methods may be used to determine the attenuation including max(aₖ). One method for attenuation estimation is:

$$\frac{1}{2 d {σ}^{2}} * ( {f}_{0} - \frac{{\sum }_{k = 0}^{p - 1} \vert {b}_{k} \vert {θ}_{k}}{{\sum }_{k = 0}^{p - 1} \vert {b}_{k} \vert} ) \qquad ( 42 )$$

where θₖ is the phase associated with aₖ, f₀ is the center frequency of the transmitted pulse, d is the scan depth and σ is related to the bandwidth of the original signal.

System Operation and Speckle Noise Reduction Process

With further regard to FIG. 3, a flow diagram of an illustrative input signal collection process 300 is shown. The process 300 may include transmitting and receiving a pulsed ultrasound signal via a pulsed ultrasound transmitter and receiver. Frequencies other than ultrasound frequencies may be utilized with transmitters and receivers configured for those frequencies may be utilized depending on the type of system and measurements to be made. At step 304, because the pulsed ultrasound signal is a time domain signal, a Fourier transform, such as an fast Fourier transform (FFT) or discrete Fourier transform (DFT) being executed on a processing unit, may be utilized to convert the received time domain pulsed ultrasound signal to the frequency domain, as understood in the art. Other domain transforms may be utilized in accordance with the principles of the present invention.

At step 306, rather than using a pulsed ultrasound signal, a stepped frequency continuous wave signal may be transmitted and received through a SFCW ultrasound transmitter and receiver. Such a transmitter and receiver allows for sampling in the frequency domain directly, but is a slower process as each stepped frequency over a frequency range that defines a bandwidth of the transmitter and receiver is to be sampled. Such a sampling, therefore, requires an operator to maintain a fixed position, such as 1/10th of a second or longer depending on the bandwidth of the measurement and number of frequency steps to be made. Resulting from the Fourier transform 304 or SFCW ultrasound receiver 306 is a number of complex frequency samples. As shown in graph 308,a number of complex frequency samples 310a-310n (N-samples) over a frequency band fₘᵢₙ to fₘₐₓ with a Δf between each of the stepped frequencies. Again, and as provided by FIG. 3, either a time domain with a Fourier transform or frequency domain sampling may be utilized to provide complex frequency samples for use in performing the resampling process 204 of FIG. 2, and as further described herein with respect to FIGS. 7-11.

With regard to FIG. 4, a flow diagram of the illustrative resampling process 400 of FIG. 2 is shown. The process 400 may include collecting a set of complex frequency samples (e.g., N samples) over a certain frequency range, fₘᵢₙ to fₘₐₓ. As shown, graphs 402a-402M-1, which represent a number of resampling iterations to be performed, each of a subset of complex frequency samples being selected and another subset of complex frequency samples (complement of the selected subset of complex frequency samples) not being selected. In one embodiment, the resampling of the subsets is performed using random resampling (i.e., complex frequency samples are randomly selected from among the set of complex frequency samples). Other selection processes that are random, pseudo random, and/or non-random may be utilized in selecting subsets of complex frequency samples. The selection of the subsets may select from among all or substantially all of the available complex frequency samples over the frequency range, thereby avoiding reduction of bandwidth and resolution, for example.

Domain transforms 404a-404M-1 (collectively 404) that convert from the frequency domain to another domain, such as the time domain, may be utilized. In one embodiment, the transforms 404 are separate transforms being executed in parallel on one or more processors. Alternatively, the transforms 404 are a single transform that performs the transforms in a serial manner as the resampling is performed. As previously described, a signal merging module 406 may be used to merge the subsets of complex frequency samples. The signal merging module 406 may utilize one or more statistical measure, including average, median, Lee, weighted average, trimmed mean, geometric mean, and so on. As shown in graph 408, from the process 400, a resulting A-scan 410 shows a low noise floor 412 relative to signal components 414 indicative of objects of interest being measured, which provides for high SNR and low speckle noise. Other noncoherent noise sources may also be reduced through the process 400.

With regard to FIG. 5, a more extensive flow diagram of a process 500 for using resampling as provided in FIG. 4 that includes combining both a sample signal and complement of the sample signal to provide a higher resolution with better SNR than possible without using the complement of the sample signal is shown. As previously described, the process 500 includes a complement of the subsampled Fourier samples for each of the M subsamples. As shown, the resampling includes (i) multiple subsets 504a-504M-1 (collectively 504) of a set of complex frequency samples and (ii) multiple complement subsets 506a-506M-1 (collectively 506) of the set of complex frequency samples. The subsets 504 and complement subsets 506 may be created using random, pseudo random, and/or non-random processes for selecting the subsets of complex frequency samples from the set of complex frequency samples. In the same manner as provided in FIG. 4, inverse transforms 508a-508M-1 and 510a-510M-1 may be used to convert the frequency domain subsets into the time domain or spatial domain. Envelope detectors 512a and 512b may be used to obtain time envelopes for the subset 504 and complement subset 506 of the complex frequency samples. Signal merging modules 514a and 514b, too, may be used for generating statistical measures of the subset 504 and complements subset 506 within the time envelope generated by the respective envelope detectors 512a and 512b. A signal merging module 514c may merge merged signals from the signal merging modules 514a and 514b. It should be understood that the envelope detectors 512a and 512b, and that signal merging modules 514a, 514b, and 514c may be the same or different modules executing on one or more processing units.

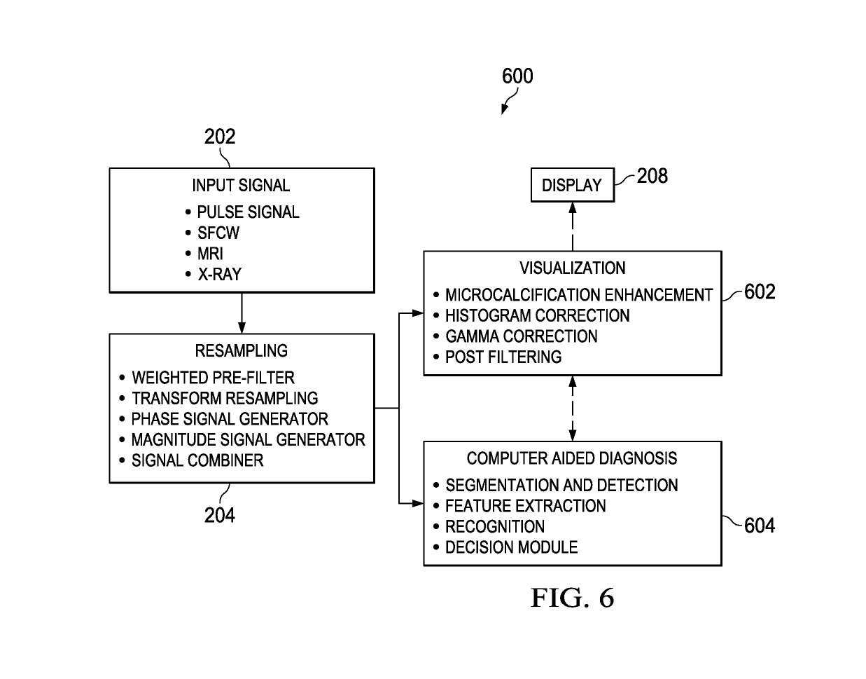

With regard to FIG. 6, a flow diagram of illustrative sub-processes 600 that may be utilized to improve SNR of an image or communications process in accordance with the principles of the present invention is shown. The sub-processes 600 may be used to form a measurement system that produces reduced speckle noise and sharper images, for example. The sub-processes 600 may include receiving an input signal 202. As shown, the input signal 202 may be a pulsed signal, stepped frequency continuous wave (SFCW) signal, magnetic resonance imaging (MRI) signal, x-ray signal, or otherwise (e.g., sonar, radar, etc.). The input signal 202 may be received using conventional hardware and/or software, as understood in the art.

A resampling sub-process 202 may include a variety of functions, including a weighted pre-filter function, transform resampling function, phase signal generator, magnitude signal generator, and signal combiner, as provided at least in FIGS. 7-11.

A visualization sub-process 602 may be configured with a number of functions, including microcalcification enhancement, histogram correction, gamma correction, and post filtering. These visualization functions may assist an operator in better visualizing features or objects of interest in an ultrasound or other image. In the case that the measurement is not an imaging function, such as an audio function, then other functions to assist an operator better hear or otherwise sense sampled signal features among noise within the samples. Output from the visualization sub-process 602 may be displayed on display 208. The output may be highlighting of features, altering of color of noise, or otherwise adjusting a sample image that may assist an operator in inspecting objects of interest within an image.

A computer aided diagnosis sub-process 604 may be configured with a number of functions, including segmentation and detection, feature extraction, feature recognition, and decision module. The functions of the computer aided diagnosis sub-process 604 may assist the operator by automatically identifying features within an image (or other measurement signal) and, possibly, determining what type of features are being automatically identified.

With regard to FIG. 7, a flow diagram of an illustrative resampling overview process 700 inclusive of sub-processes utilizing the principles of the present invention is shown. The resampling overview process 700 may include an input signal receive sub-process 702, where the input signal may be in the time domain (e.g., pulsed signal) or frequency domain (e.g., SFCW signal). Use of sampling in the time domain may be a faster process than in the frequency domain, as previously described.

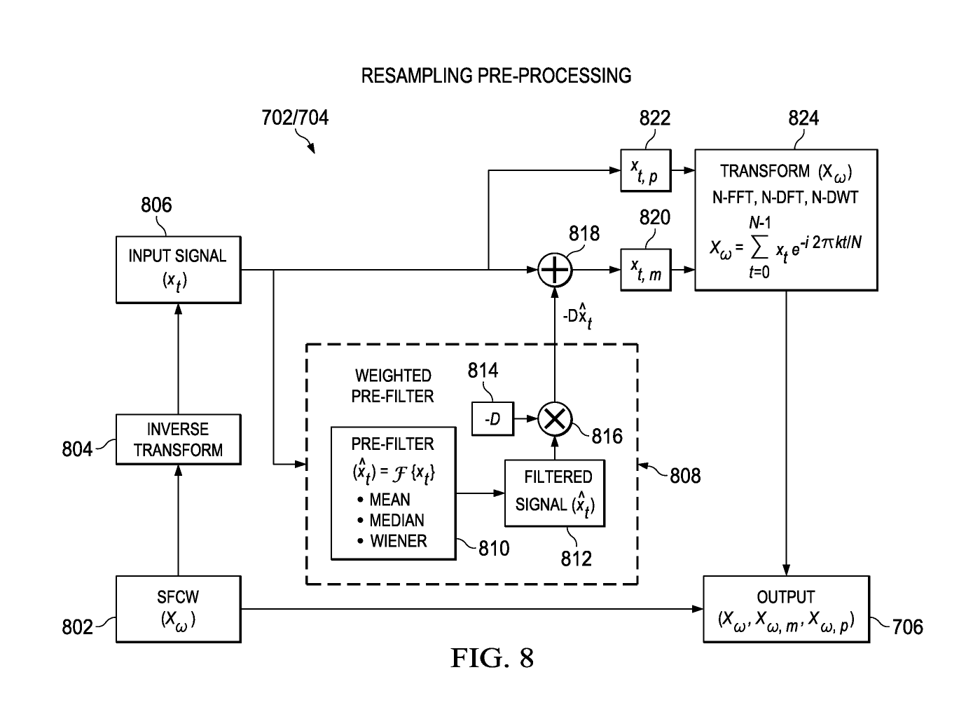

At step 704, a preprocessing sub-process 704 may be performed. In the event of the input signal being in the frequency domain, a conversion from the frequency to time domain may be performed using an inverse FFT or other transform function. Other domains and transforms may be utilized in accordance with the principles of the present invention. An optional offset using a weighted pre-filter may be utilized prior to converting, as is provided in FIG. 8. The pre-processing sub-process 704 may also transform a time domain signal to the frequency domain using an N-length transform to generate an output signal Xω 706.

With regard to FIG. 8, a flow diagram of illustrative a resampling pre-processing sub-process 702/704 of input signals of FIG. 7 is shown. The sub-process 704 may include receiving either a SFCW Xω 802, in which case an inverse Fourier transform (e.g., IFFT) may be performed to convert the input signal from the frequency to time domain. Alternatively, if the input signal Xt 806 is a time domain signal, then a transform is unnecessary. Optionally, a weighted pre-filter may be utilized to filter the input signal 806. The weighted pre-filter 808 may be used to remove bias or other measurement artifacts from the raw input signal 806. The weighted pre-filter may include a pre-filter 810 that is a function of the measured input signal formed of a set of complex frequency signals. The function may be a mathematical statistical function or otherwise. For example, the function may be a mean, median, Wiener filter, or otherwise. A filtered signal 812 may be output from the pre-filter 810, and an inverted weight 814, in this case—D, may be applied to the filtered signal 812 using multiplier 816. The inverted and weighted filtered signal may offset the input signal 806 using a summer 818 along a magnitude channel to produce pre-filtered signal x_{t,m}. Phase of the input signal xₜ is not filtered, and is input as x_{t,p} 822 to transform 824. The transform 824 may be any transform, such as N-FFT, N-DFT, N-DWT, etc., that converts the time domain signal to an output signal 706 in the frequency domain of the resampling pre-processing. If the input signal 802 is not to be pre-filtered, then the input signal 802 may become the output signal 706.

Continuing with FIG. 7, a mask generation sub-process 708 may be configured to create a mask that may be used for selecting a subset of complex frequency samples from a set of complex frequency samples across an entire bandwidth of the complex frequency samples. More specifically, with regard to FIG. 8, a flow diagram of the resampling mask generation sub-process 708 of FIG. 7 for generating random samples of the pre-processed input signals of FIG. 8 is shown. The input signal 706 is the output of the resampling pre-processing sub-process 702/704 of FIG. 8, and is the frequency domain inclusive of frequency, magnitude, and phase information. A K-time loop 902 may include a random mask generator 904 configured to generate a random mask 905 of length N. The random mask 905 may be a series M₁-M_{K} of arrays or matrix formed of N binary numbers (i.e., 0s and 1s) created by a random number generator, as understood in the art. In addition, a random mask complement generator 906 may be configured to generate a complement random mask 907 of the random mask 905. The generator 906 may be implemented in a variety of ways for generating digital complement matrices, as understood in the art.

The random mask 905 and complement random mask 907 may be used as multipliers of the input signal 706 to generate outputs 710a and complement outputs 710b, both of which are inclusive of magnitude and phase information. As shown, by using binary values, any data of the input signal 706 that is multiplied by a 0 becomes a 0 in the outputs 710a and 710b. The entire spectrum of the input signal 706 is maintained, thereby maintaining full bandwidth and, thus, resolution is not degraded. However, by resampling in a random manner, speckle noise can be distinguished from objects of interest to allow for reduction or elimination of the speckle noise.

Although the use of random masks may be utilized by the resampling mask generation sub-process 708, other pseudo random and non-random masks may alternatively be utilized in accordance with the principles of the present invention. As understood in the art, random number generators on computers have limitations as to their randomness. However, the principles of the present invention are not dependent upon the overall randomness of a random number generator. For example, a predetermined set of masks may be applied to the input signal 706 and achieve a similar end-result with regard to reducing or eliminating speckle noise.

With further regard to FIG. 7, an inverse transform sub-process or module 712 may be performed on the output signals 710a and 710b from the mask generator 708. The inverse transform may be an inverse fast Fourier transform (IFFT), inverse discrete Fourier transform (IDFT), inverse discrete wavelet transform (IDWT), and so on, as understood in the art to transform the output signals 710a and 710b from the frequency domain to another domain, such as the time domain or spatial domain. From the inverse transform sub-process 712, two outputs 714a and 714b in the different domain may be generated, where the two outputs 714a and 714b are complements of one another as a result of the complements of output signals 710a and 710b. That is, the inverse transform sub-process 712 transforms each of the output signals 710a and 710b independent of one another.

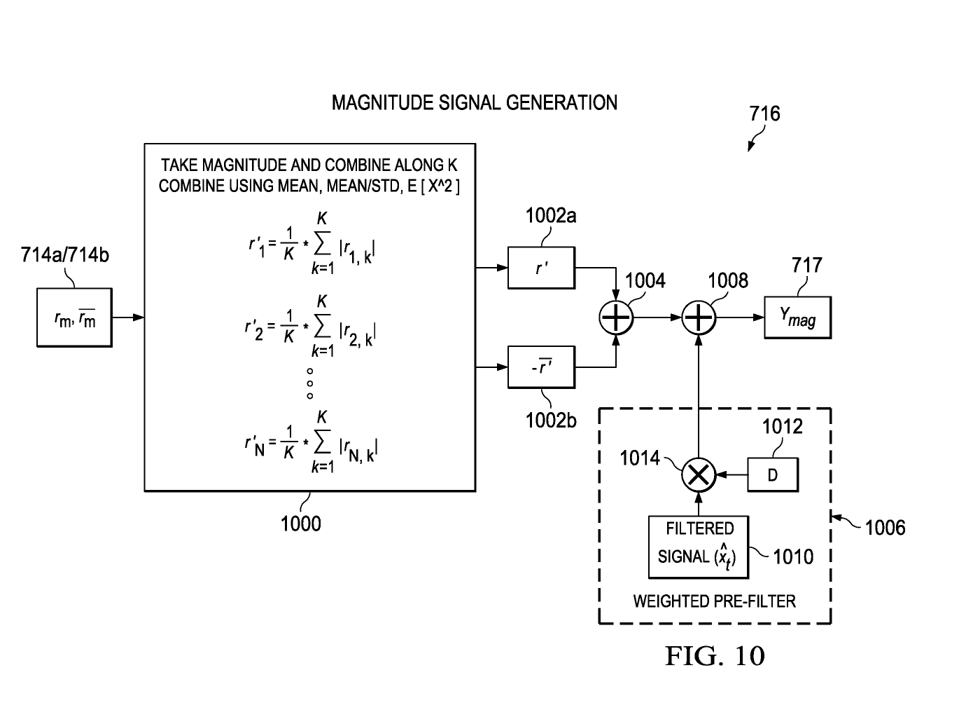

The two outputs 714a and 714b may be inputs to a magnitude signal generator sub-process or module 716 that is configured to (i) take a magnitude, (ii) combine K by N signals, (iii) combine the complement, and (iv) add weighted pre-filter. More specifically, and with regard to FIG. 10, a flow of an illustrative magnitude signal generation sub-process 716 of FIG. 7 is shown. The output signals 714a and 714b may be submitted to a magnitude generator module 1000 configured to generate an average magnitude for each pixel and complement of each pixel by combining along each array or row (i.e., pixel) of the output signals 714a and 714b. Other statistics, such as mean/standard deviation (STD), variance, or other statistical metric may be generated, as understood in the art. Outputs from the magnitude generator module 1000 may be magnitude and complement magnitude 1002a and 1002b of the input signals 714a and 714b. A subtraction of the complement of the magnitude of each pixel by summer 1004. In one optional embodiment, a weighted pre-filter 1006 may be utilized to subtract a local mean of an image via summer 1008. The weighted pre-filter may generate a filtered signal 1010 and apply a weight 1012 using multiplier 1014. An output magnitude signal 717 may be produced by the magnitude signal generation sub-process 716.

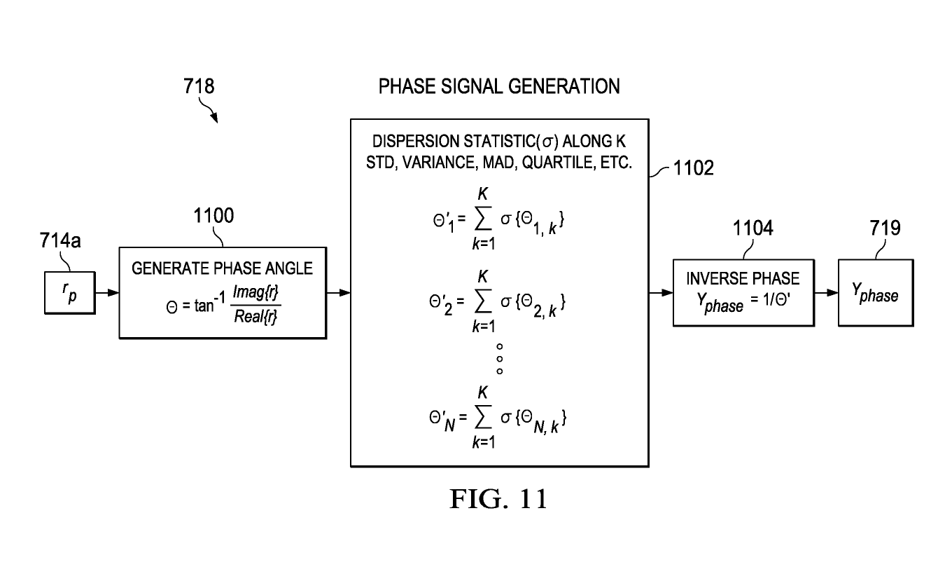

FIG. 7 further includes a phase signal generation sub-process 718 configured to (i) compute phase, (compute dispersion statistics along K), and (iii) take an inverse of the output signal 714a (note that the complement output signal 714b is not used by the phase signal generation sub-process 718. FIG. 11 provides a flow diagram of the illustrative phase signal generation sub-process 718 of FIG. 7. A phase angle generator 1100 generates a phase angle by using the real and imaginary portions of each pixel. A dispersion statistic generator 1102 may be configured to generate dispersion statistic(s) of each pixel. The generator 1102 may generate other statistics, including standard deviation, variance, median absolute deviation (MAD), quartile, etc. Standard deviation of speckle noise is large for any given pixel, while standard deviation of content is small at any given pixel. So, if a standard deviation is determined to be large at a pixel, then it may be determined that the pixel includes speckle noise and the system may be configured to reduce the speckle noise at that pixel. In the case of determining that a pixel has identified actual content data (e.g., object of interest), then amplification may be performed for that pixel to preserve the signal. Other statistical measures may be utilized to help identify and distinguish pixel noise from content data within the received signal (e.g., ultrasound signal). An inverse phase generator 1104 may be used for display purposes, where an inverse of a large standard deviation is small and an inverse of a small standard deviation is large, such that those parameters may be used to adjust amplitude of each pixel (e.g., noisy pixel magnitude can be darkened, and content pixel can be brightened). Output from the inverse phase generator 1104 is an output phase signal 719.

As further provided in FIG. 7, a combiner sub-process 720 may be configured to (i) combine magnitude signal 717 and phase signal 719, (ii) generate geometric mean, and (iii) generate mean, as understood in the art, and generate a combined output signal 722 of the combined magnitude and phase signals 717 and 719. It should be understood that the combiner sub-process 720 may be configured to generate other statistical parameters, as understood in the art, for use in adjusting and/or displaying an image represented by the magnitude signal 717 and/or phase signal 719. In the case where the system is configured to process non-image signals, such as audio signals, the same, similar, analogous, or different statistical measurements may be generated for use in adjusting output of the measured signals.

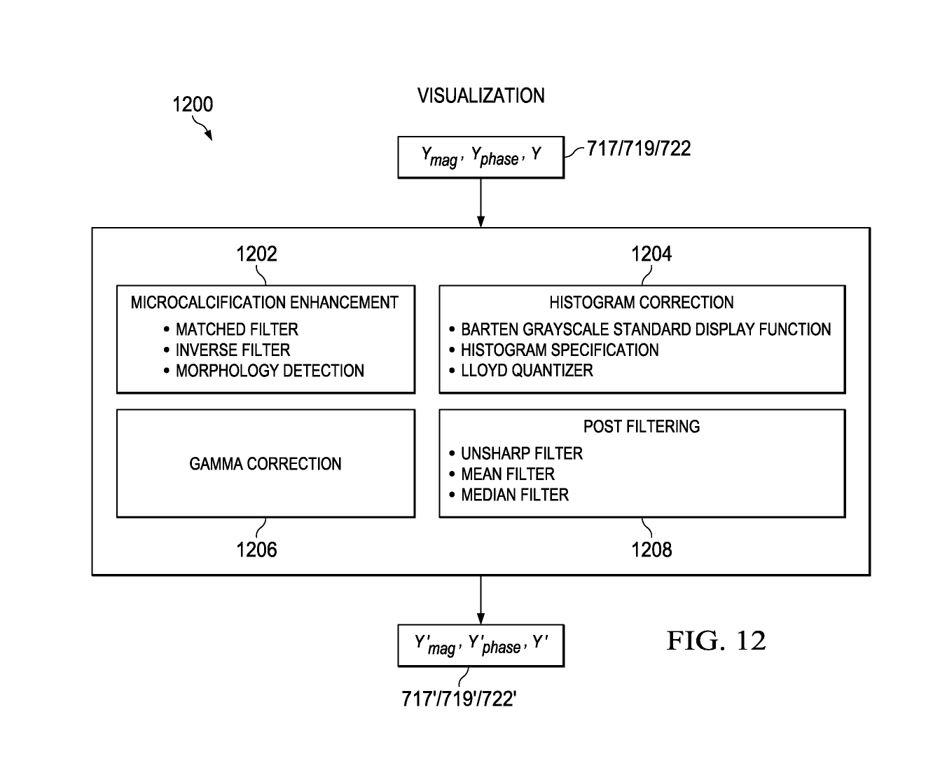

With regard to FIG. 12, an illustrative visualization sub-process 1200 that may be used to improve visualization of output signals for an operator of a measurement system is shown. The visualization sub-process 1200 may include receiving the output magnitude signal 717, output phase signal 719, and combined output signal 722. A microcalcification enhancement module 1202 may be configured with a (i) matched filter, (ii) inverse filter, and/or (iii) morphology detection algorithm to enhance the specific object of interest of microcalcification. The matched filter may include use of a small Gaussian filter representative a model microcalcification so that any outputs from the matched filter above a threshold would indicate that a microcalcification feature is identified, while outputs from the matched filter below the threshold would indicate that a non-microcalcification feature is not identified.

Also included in the visualization sub-process 1200 is a histogram correction module 1204 that may be configured with a (i) Barten grayscale standard display function, (ii) histogram specification, and/or (iii) Lloyd quantizer. Alternative and/or different histogram correction functionality may be utilized in accordance with the principles of the present invention. A gamma correction module 1206 and post filtering module 1208 may also be included as part of the visualization sub-process 1200, where the post filtering module 1208 may include an (i) unsharp filter, (ii) mean filter, (iii) median filter, or (iv) any other image adjustment filter, as understood in the art. It should be understood that other image or other processing functionality may be utilized as part of the visualization sub-process 1200 as understood in the art to aid an operator of an ultrasound or other imaging system inspect features captured in an image.

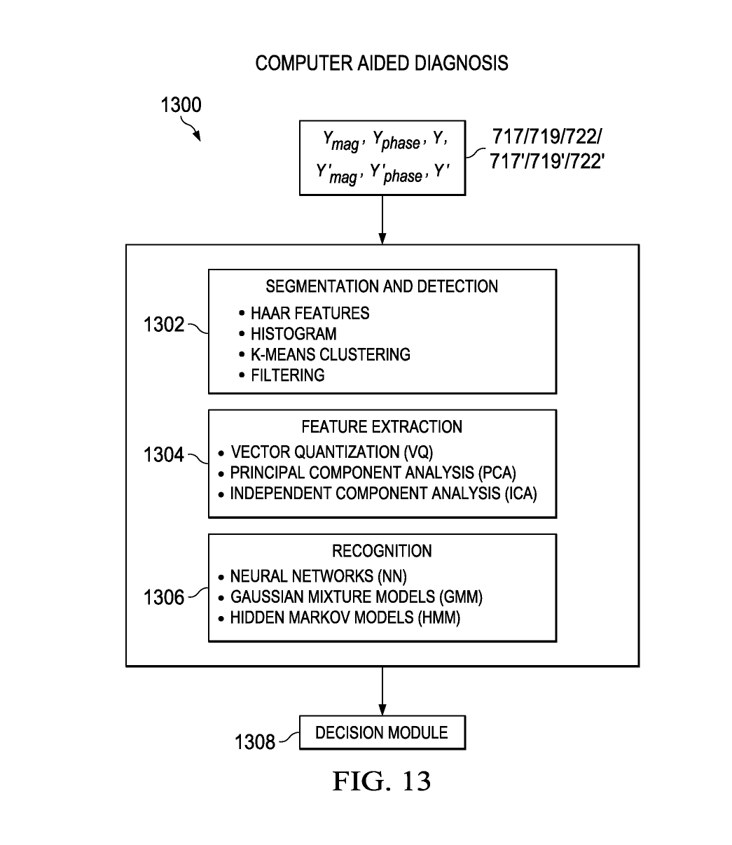

With regard to FIG. 13, a block diagram of an illustrative computer aided diagnosis sub-process 1300 for assisting an operator in determining features included within a measurement (e.g., image) captured by a measurement system is shown. The output signals 717, 719, 722 and their complements 717′, 719′, 722′ may be input to the computer aided diagnosis sub-process 1300. A segmentation and detection module 1302 may be used to segment and detect structures in an image, and may include (i) HAAR features, (ii) histogram function, (iii) K-means clustering function, and (iv) filtering functions, as understood in the art. A feature extraction module 1304 may be used to extract structures in an image, and include (i) vector quantization (VQ) functionality, (ii) principal component analysis (PCA), and (iii) independent component analysis (ICA). Other and/or different feature extraction functionality may be utilized in accordance with the principles of the present invention. A recognition module 1306 may utilized (i) neural networks (NN), (ii) Gaussian mixture models (GMM), and (iii) hidden Markov models (HMM) for use in recognizing particular structures, such as microcalcification structures, as a result of sharpness, shapes, or otherwise of features of the microcalcification or other structures. Other artificial intelligence or recognition functionality may be included in the recognition module 1306.

A decision module 1308 may be configured to automatically make a decision based on the functionality performed by the modules 1302, 1304, and 1306. Such decision may include generating a notification indicative of the system determining that a certain structure, such as microcalcification or tumor, is detected. In one embodiment, decision module 1308 may use one or more threshold values, such as percentage(s), to generate one or more decisions (e.g., 90% chance that microcalcification is detected). The sub-process 1300 may assist an operator in helping to determine or confirm that certain features or structures are captured by the measurement system.

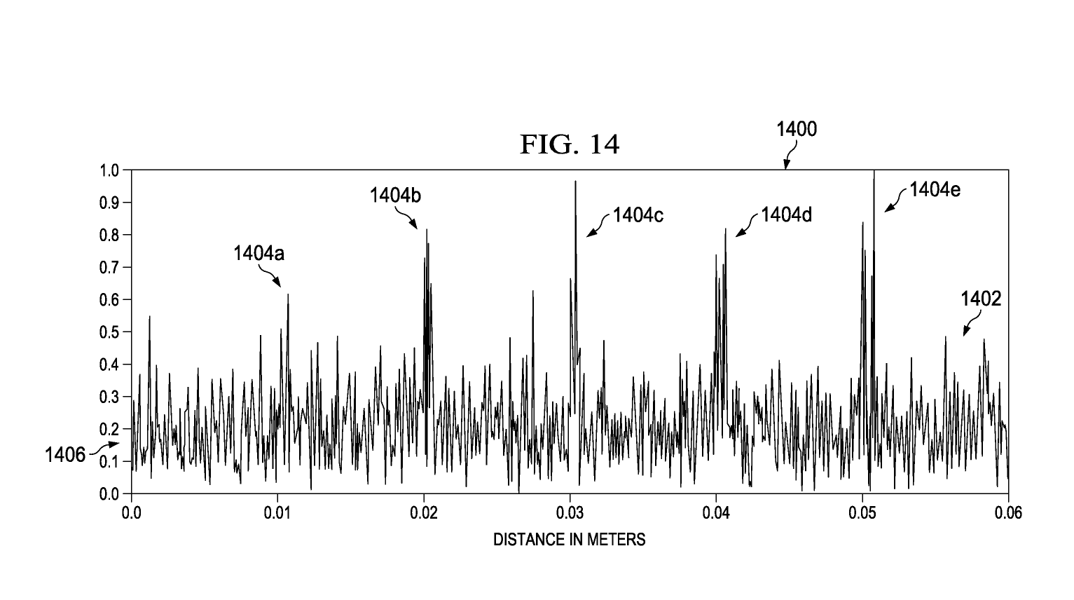

With regard to FIG. 14, a graph 1400 showing an illustrative raw ultrasound A-scan 1402 inclusive of microcalcifications and noise collected from an ultrasound system is shown. The raw ultrasound A-scan 1402 inclusive of signal features 1404a-1404e (collectively 1404) and noise 1406 (e.g., speckle noise).

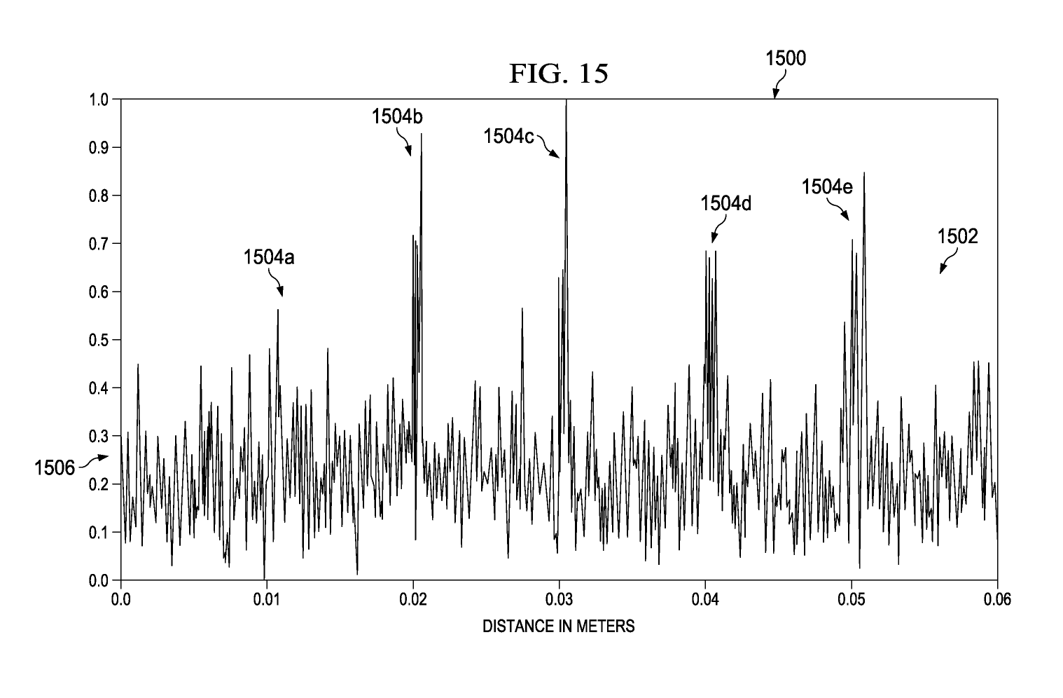

With regard to FIG. 15, a graph 1500 showing the measured signal of FIG. 14 after being processed (processed A-scan signal 1502) using conventional frequency compounding techniques is shown. As shown, the processed A-scan 1502 of the A-scan 1402 has an improved SNR, as the signal features 1504a-1504e (collectively 1504) is more separated from the processed noise 1506.

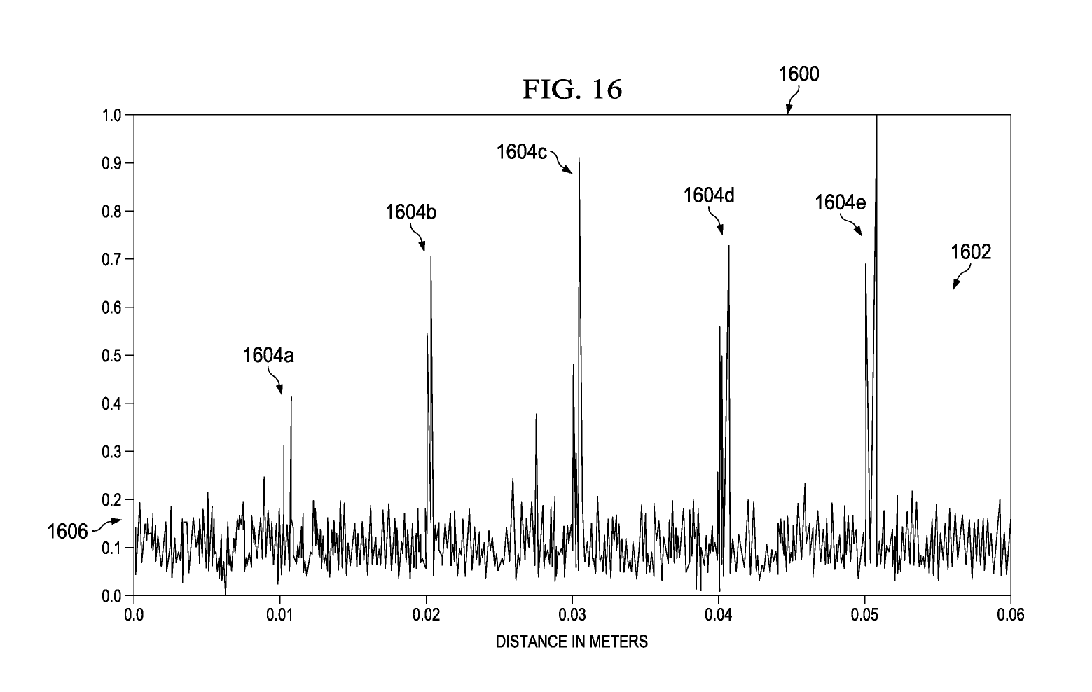

With regard to FIG. 16, a graph 1600 showing the measured signal of FIG. 14 after being processed (processed A-scan signal 1602) using the resampling process provided in FIGS. 7-11. The processed A-scan signal 1602 is shown to have the same or slight improved signal features 1604a-1604e and lower noise 1606. As previously described, speckle noise is reduced as a result of processing the raw A-scan signal over its entire bandwidth as opposed to using compounding techniques and processing multiple, narrow bandwidths of the A-scan.

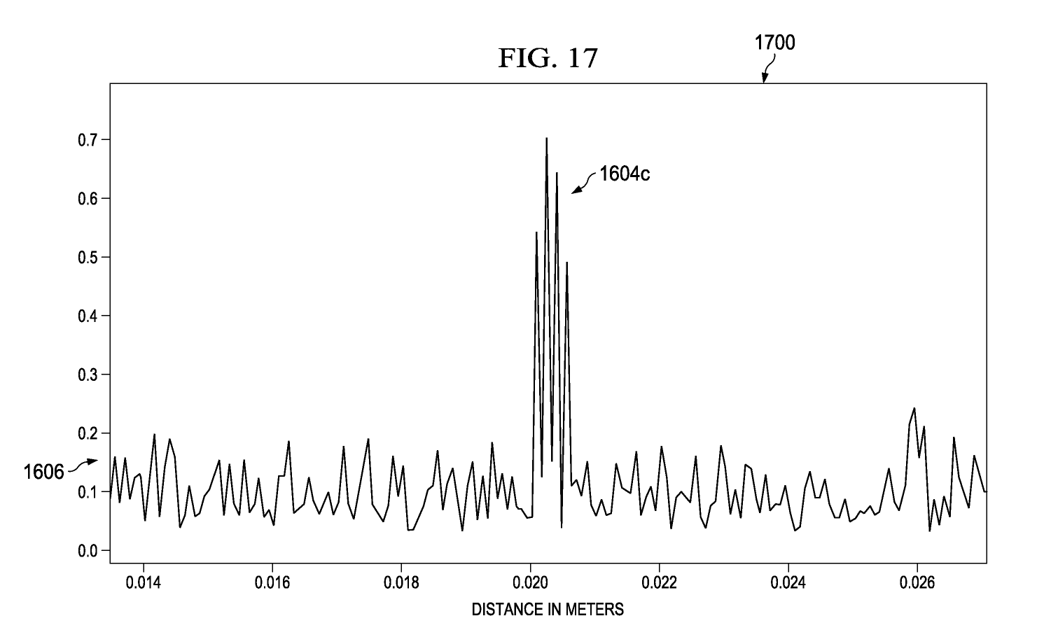

With regard to FIG. 17, a graph 1700 showing a zoomed-in view of a portion of the processed A-scan of FIG. 16 is shown. The signal feature 1604c is shown with sufficient resolution to identify multiple features, such as jagged edges, of an object of interest. Noise 1606 is also shown to be at a relatively low level (e.g., at or below a certain noise floor).

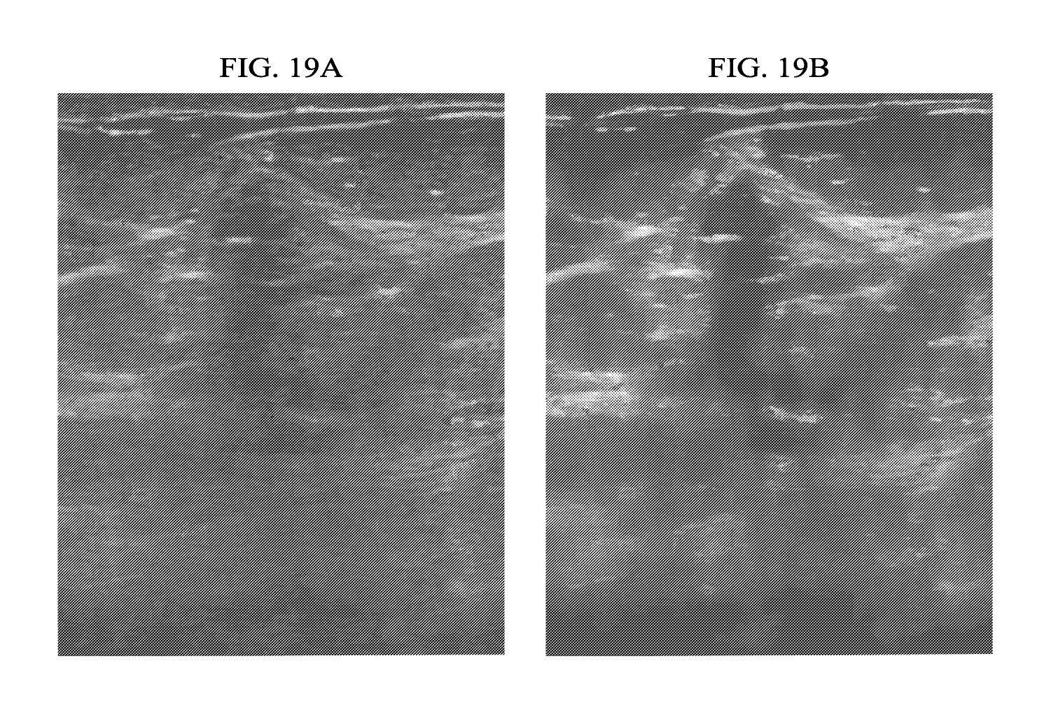

With general regard to FIGS. 19A and 19B, 20A and 20B, 21A and 21B, and 22A and 22B (collectively FIGS. 19, 20, 21, and 22, respectively) B-scan images of a raw ultrasound signal and speckle noise corrected ultrasound signal showing how speckle noise is reduced and lesion boundaries are clearer are shown. The grainy appearance (salt and pepper type noise) on each of the left images (i.e., FIGS. 19A, 20A, 21A, 22A) is speckle, where much of the speckle noise is removed in the images on the right (i.e., FIGS. 19B, 20B, 21B, and 22B) resulting in smooth areas. The remaining small details on the corrected A-scans are due to actual tissue features including microcalcifications and other details in the tissue and lesions.

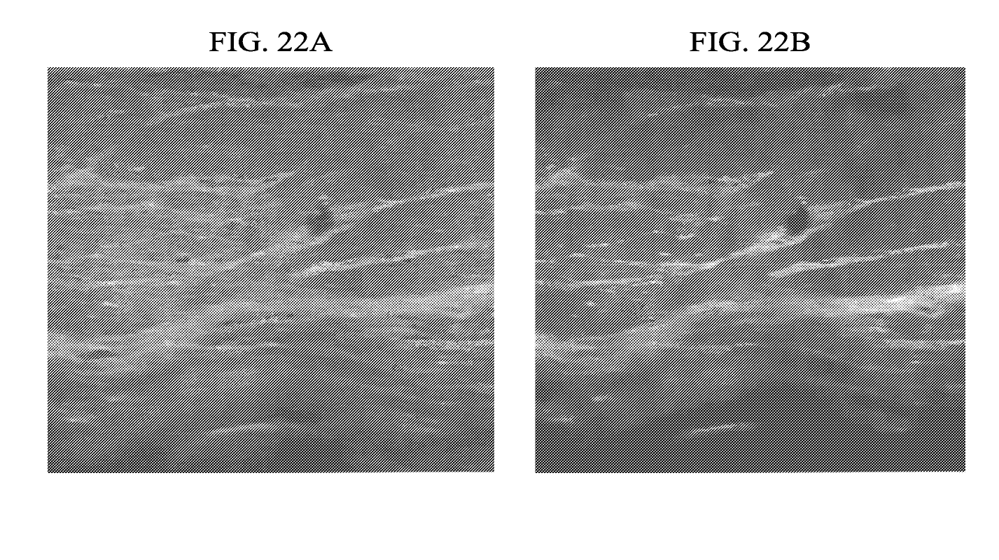

FIGS. 19-21 show lesions that are large, dark, and irregularly shaped regions towards the left of the images, while FIG. 22 shows a lesion being a small dark area slightly right and up from the center of the image.

With specific regard to FIGS. 19-22, FIGS. 19A and 19B are B-scan images of an ultrasound signal optimized for capture and viewing by a trained radiologist (FIG. 19A) and speckle noise corrected ultrasound signal (FIG. 19B) showing a suspicious lesion (dark area) in breast tissue that was assessed as BI-RADS® 4 or 5 by a trained radiologist and recommended for biopsy. Note the reduced speckle noise (graininess) and clearer tissue structure and small details that are visible in the speckle noise corrected image (FIG. 19B);

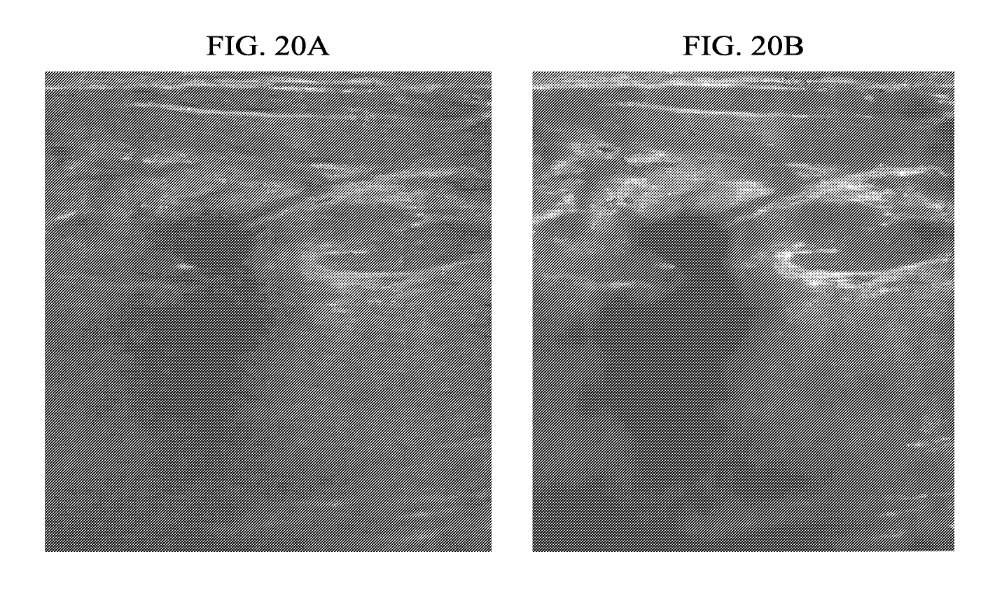

FIGS. 20A and 20B are B-scan images of an ultrasound signal optimized for capture and viewing by a trained radiologist (FIG. 20A) and speckle noise corrected ultrasound signal (FIG. 20B) showing a suspicious lesion (dark area) in breast tissue that was assessed as BI-RADS® 4 or 5 by a trained radiologist and recommended for biopsy. Note the reduced speckle noise (graininess) and clearer tissue structure and small details that are visible in the speckle noise corrected image (FIG. 20B);

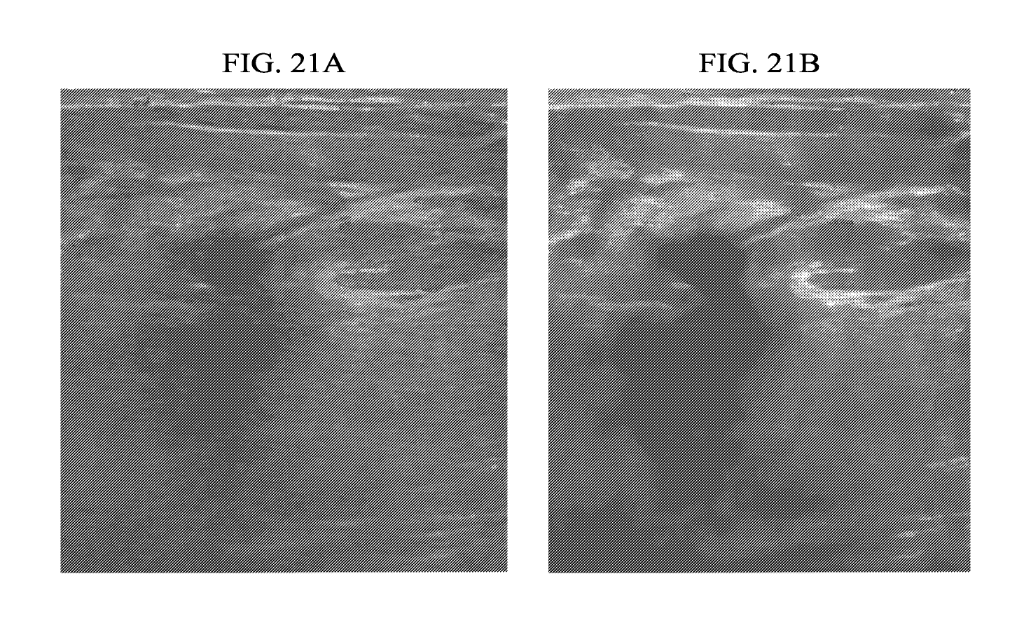

FIGS. 21A and 21B are B-scan images of an ultrasound signal optimized for capture and viewing by a trained radiologist (FIG. 21A) and speckle noise corrected ultrasound signal (FIG. 21B) showing a suspicious lesion (dark area) in breast tissue that was assessed as BI-RADS® 4 or 5 and recommended for biopsy. Note the reduced speckle noise (graininess) and clearer tissue structure and small details that are visible in the speckle noise corrected image (FIG. 21B); and

FIGS. 22A and 22B are B-scan images of an optimized ultrasound signal measured with a state-of-the-art ultrasound device and speckle noise corrected ultrasound signal showing a lesion in breast tissue. Note the reduced speckle noise (graininess) and clearer tissue structure and small details that are visible in the speckle noise corrected image (FIG. 22B).

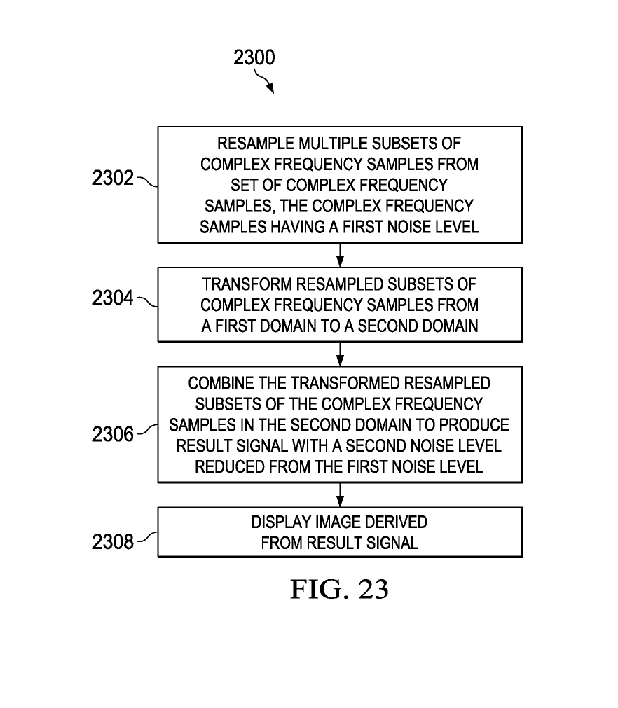

With regard to FIG. 23, a flow diagram of an illustrative process 2300 for resampling an ultrasound signal to reduce speckle noise is shown. The process 2300 starts at step 2302, where multiple subsets of complex frequency samples are resampled from a set of complex frequency samples. The complex frequency samples have a first noise level. At step 2304, resampled subsets of complex frequency samples may be transformed from a first domain to a second domain. At step 2306, the transformed resampled subsets of the complex frequency samples may be combined in the second domain to produce a result signal with a second noise level reduced from the first noise level. An image derived from the result signal may be displayed. As previously described, the principles of the present.

In one embodiment, the resampled subsets of complex frequency samples include subsets of complex frequency samples that are randomly selected from the set of complex frequency samples. The combined resampled subsets may be combined in the time domain. The combining may be combined noncoherently. Magnitude of each transformed resampled subset of complex frequency sample points may be combined independent of combining phase of each transformed resampled subset of complex frequency sample points. Phase of each transformed resampled subset of complex frequency sample points may be combined independent of combining magnitude of each transformed resampled subset of complex frequency sample points. At least one statistical measure of the transformed resampled subsets of the complex frequency samples may be generated. The statistical measure(s) may include a standard deviation and a mean. A second statistical measure may be generated by dividing mean by standard deviation. A variance and standard deviation may be generated and used to adjust the image. In one embodiment, the ultrasound signal is a pulsed ultrasound signal. Other non-ultrasound image signals (e.g., x-ray, MRI, etc.), non-image signals (e.g., audio, RF, etc.) may be utilized in accordance with the principles of the present invention.

The previous detailed description is of a small number of embodiments for implementing the invention and is not intended to be limiting in scope. One of skill in this art will immediately envisage the methods and variations used to implement this invention in other areas than those described in detail. The following claims set forth a number of the embodiments of the invention disclosed with greater particularity.

What is claimed:

1. A method of processing an ultrasound signal, said method comprising:
in response to receiving a set of complex frequency samples of the ultrasound signal inclusive of content data and noise at a first noise level and being used to image an anatomical region of a body, resampling a plurality of subsets of complex frequency samples from the set of complex frequency samples;
transforming the resampled subsets of complex frequency samples from a first domain into a second domain;
combining the transformed resampled subsets of complex frequency samples in the second domain to produce a result signal with a second noise level reduced from the first noise level; and
displaying an image derived from the result signal.

2. The method according to claim 1, wherein the resampled subsets of complex frequency samples include subsets of complex frequency samples that are randomly selected from the set of complex frequency samples.

3. The method according to claim 1, wherein combining includes combining resampled subsets in the time domain.

4. The method according to claim 1, wherein combining includes noncoherently combining the transformed resampled subsets.

5. The method according to claim 1, wherein combining includes combining magnitude of each transformed resampled subset of complex frequency sample points independent of combining phase of each transformed resampled subset of complex frequency sample points.

6. The method according to claim 1, wherein combining includes combining the phase of each transformed resampled subset of complex frequency sample points independent of combining magnitude of each transformed resampled subset of complex frequency sample points.

7. The method according to claim 1, wherein combining includes generating at least one statistical measure of the transformed resampled subsets of the complex frequency samples.

8. The method according to claim 7, wherein generating includes generating at least one of a standard deviation and a mean.

9. The method according to claim 8, further comprising generating a second statistical measure by dividing mean by standard deviation.

10. The method according to claim 7, wherein generating includes generating variance and standard deviation.

11. The method according to claim 1, wherein the ultrasound signal is a pulsed ultrasound signal.

12. The method according to claim 1, further comprising correcting depth and frequency attenuation of the ultrasound signal by generating an attenuation matrix A and scaling each coefficient by an exponential attenuation factor.

13. The method according to claim 1, further comprising performing a frequency domain regression analysis on the ultrasound signal to identify the content data and noise data, thereby providing for the noise data to be reduced.

14. The method according to claim 1, further comprising performing a time domain regression on the ultrasound signal to identify attenuation due to different tissue types, thereby providing for characterization of tissue being measured.

15. A system for processing an ultrasound signal, said system comprising:
a processing unit;
a memory configured to store data, and in communication with said processing unit, said processing unit configured to:
in response to receiving a set of complex frequency samples of the ultrasound signal inclusive of content data and noise at a first noise level and being used to image an anatomical region of a body, resample a plurality of subsets of complex frequency samples from the set of complex frequency samples;
transform the resampled subsets of complex frequency samples from a first domain into a second domain;
combine the transformed resampled subsets of complex frequency samples in the second domain to produce a result signal with a second noise level reduced from the first noise level; and
display an image derived from the result signal.

16. The system according to claim 15, further comprising:
a signal generator configured to generate the ultrasound signal
a transducer in communication with said signal generator, and configured to convert the ultrasound signal into an incident ultrasound signal;
a receiver in communication with said processing unit, and configured to receive a reflected ultrasound signal.

17. The system according to claim 15, wherein the resampled subsets of complex frequency samples include subsets of complex frequency samples that are randomly selected from the set of complex frequency samples.

18. The system according to claim 15, wherein said processing unit, in combining the transformed resampled subsets of complex frequency samples, is further configured to combine resampled subsets in the time domain.

19. The system according to claim 15, wherein said processing unit, in combining the transformed resampled subsets of complex frequency samples, is further configured to noncoherently combine the transformed resampled subsets.

20. The system according to claim 15, wherein said processing unit, in combining the transformed resampled subsets of complex frequency samples, is further configured to combine magnitude of each transformed resampled subset of complex frequency sample points independent of combining phase of each transformed resampled subset of complex frequency sample points.

21. The system according to claim 15, wherein said processing unit, in combining the transformed resampled subsets of complex frequency samples, is further configured to combine the phase of each transformed resampled subset of complex frequency sample points independent of combining magnitude of each transformed resampled subset of complex frequency sample points.

22. The system according to claim 15, wherein said processing unit, in combining the transformed resampled subsets of complex frequency samples, is further configured to generate at least one statistical measure of the transformed resampled subsets of the complex frequency samples.

23. The system according to claim 22, wherein said processing unit, in generating the at least one statistical measure, is further configured to generate at least one of a standard deviation and a mean.

24. The system according to claim 23, wherein said processing unit is further configured to generate a second statistical measure by dividing mean by standard deviation.

25. The system according to claim 22, wherein said processing unit, in generating the at least one statistical measure, is further configured to generate variance and standard deviation.

26. The system according to claim 15, wherein the ultrasound signal is a pulsed ultrasound signal.

27. The method according to claim 15, further comprising correcting depth and frequency attenuation of the ultrasound signal by generating an attenuation matrix A and scaling each coefficient by an exponential attenuation factor.

28. The method according to claim 15, further comprising performing a frequency domain regression analysis on the ultrasound signal to identify the content data and noise data, thereby providing for the noise data to be reduced.

29. The method according to claim 15, further comprising performing a time domain regression on the ultrasound signal to identify attenuation due to different tissue types, thereby providing for characterization of tissue being measured.

30. A method of processing a coherent signal, said method comprising:
in response to receiving a set of complex frequency samples of the coherent signal inclusive of content data and noise at a first noise level, resampling a plurality of subsets of complex frequency samples from the set of complex frequency samples;
transforming the resampled subsets of complex frequency samples from a first domain into a second domain;
combining the transformed resampled subsets of complex frequency samples in the second domain to produce a result signal with a second noise level reduced from the first noise level; and
outputting the result signal.

31. The method according to claim 30, wherein the signal is an ultrasound signal.

32. The method according to claim 31, wherein receiving the ultrasound signal includes receiving the ultrasound signal of an anatomical region of a body.

33. The method according to claim 30, wherein outputting the result signal includes displaying the result signal on an electronic display.

34. The method according to claim 30, further comprising generating the ultrasound signal.
